(19) United States
(12) Patent Application Publication
(10) Pub. No.: US 2012/0322153 A1
(43) Pub. Date: Dec. 20, 2012

(54) REPROGRAMMING A CELL BY ACTIVATION OF THE ENDOGENOUS TRANSCRIPTION FACTOR NETWORK

(75) Inventors: Kenneth J. Eilertsen, Baton Rouge, LA (US); Jong S. Rim, Baton Rouge, LA (US); Rachel A. Power, Zachary, LA (US); Jaroslaw Staszkiewicz, Baton Rouge, LA (US)

(73) Assignee: NUPOTENTIAL, INC., Baton Rouge, LA (US)

(21) Appl. No.: 13/390,822

(22) PCT Filed: Aug. 17, 2010
(86) PCT No.: PCT/US10/45777

§ 371 Date: Sep. 4, 2012

Related U.S. Application Data
(60) Provisional application No. 61/234,843, filed on Aug. 18, 2009.

(51) Int. Cl. C12N 5/071

(52) U.S. Cl. 435/375

(57) ABSTRACT

The invention relate to methods, compositions, and kits for reprogramming a cell. In one embodiment, the invention relates to a method comprising inducing the expression of at least one gene that contributes to a cell being pluripotent or multipotent. In yet another embodiment, the method comprises delivering a transcription factor to a cell and exposing said cell to an agent that inhibits the activity, expression, or activity and expression of a gene, which codes for a protein, or a protein involved in transcriptional repression, and selecting a cell, wherein differentiation potential has been restored to said cell. In yet another embodiment, the invention relates to a reprogrammed cell and an enriched population of reprogrammed cells that can have characteristics of an ES-like cell can be re- or trans-differentiated into various differentiated cell types.

### FIELD

Embodiments of the invention relate to the fields of cell biology, stem cells, cell differentiation, somatic cell nuclear transfer and cell-based therapeutics. More specifically, embodiments of the invention are related to methods, compositions and kits for reprogramming cells and cell-based therapeutics. The invention also relates to methods for improving the efficiency of reprogramming, and reprogrammed cell lines.

### BACKGROUND

Regenerative medicine holds great promise as a therapy for many human ailments, but also entails some of the most difficult technical challenges encountered in modern scientific research. The technical challenges to regenerative medicine include low cloning efficiency, a short supply of potentially pluripotent tissues, and a generalized lack of knowledge as to how to control cell differentiation and what types of embryonic stem cells can be used for selected therapies. While ES cells have tremendous plasticity, undifferentiated ES cells can form teratomas (benign tumors) containing a mixture of tissue types. In addition, transplantation of ES cells from one source to another likely would require the administration of drugs to prevent rejection of the new cells.

Attempts have been made to identify new avenues for generating stem cells from tissues that are not of fetal origin. One approach involves the manipulation of autologous adult stem cells. The advantage of using autologous adult stem cells for regenerative medicine lies in the fact that they are derived from and returned to the same patient, and are therefore not subject to immune-mediated rejection. The major drawback is that these cells lack the plasticity and pluripotency of ES cells and thus their potential is uncertain. Another approach is aimed at reprogramming somatic cells from adult tissues to create pluripotent ES-like cells. However, this approach has been difficult as each cell type within a multi-cellular organism has a unique epigenetic signature that is thought to become fixed once cells differentiate or exit from the cell cycle.

In 2006, Yamanaka et al. reported that introduction of four transcription factors (Oct4, Sox2, c-Myc, and Klf4) into mouse fibroblasts by retroviral infection produced cells exhibiting embryonic stem (ES) cell-like morphology and proliferation capacity, endogenous pluripotency gene expression, and restored in vitro and in vivo differentiation capacity (Cell 2006; 126:663-676). Yamanaka et al. reported generation of 160 ES cell-like colonies per 8×10⁵ mouse embryonic fibroblasts (0.1% efficiency, Table I).

In a similar fashion, Thomson et al. demonstrated that the tumor related factors, Klf4 and cMyc, could be replaced by Lin28 and Nanog without changing the reprogramming efficiency (about 0.02% efficiency) (Science, Vol. 318. no. 5858, pp. 1917-1920, 2007). However, removal of either Oct4 or Sox2 from the viral cocktail eliminated the formation of iPS colonies.

Genome-wide mapping of the four required transcription factors, Oct4, Sox2, Klf4 and c-myc, as well as five other related factors, Nanog, Dax1, Rex1, Zpf281, and Nac1, revealed specific-promoter occupancies in embryonic stem cells, providing a more detailed understanding of the regulatory transcriptional network. Among the 6,632 target gene promoters bound by the nine transcription factors, approximately 50% of the genes are occupied by only one of the nine tested transcription factors indicating that target gene promoters are co-occupied by multiple transcription factors.

The efficiencies of iPS cell production are influenced by reprogramming strategies including (1) the combination of transcription factors; (2) recipient cell type; (3) transduction methods; and (4) the presence of epigenetic modifiers. A summary of the various methodologies and the resulting reprogramming efficiencies is provided in Table I.

TABLE I
A summary of reprogramming efficiency with various methods
Reprogramming timing & efficiency byiPS strategy

| Strategy | HDAC inhibitor | Cell type | Timing & Efficiency | |
|---|---|---|---|---|
| Retroviral infection | | | | |
| 4 iPS factors (OSKM)* | | Mouse embryonic fibroblasts | ~4 weeks | 0.1% |
| 3 iPS factors (OSK) | | Mouse embryonic fibroblasts | | 0.01% |
| 4 iPS factors (OSKM) | | Human fibroblasts | | 0.02% |
| 3 iPS factors (OSK) | | Human fibroblasts | | 0.001% |
| 3 iPS factors (OSK) | VPA** | Human fibroblasts | ~2 weeks | 1% |
| 2 iPS factors (OS) | | Human fibroblasts | | No colonies |
| 2 iPS factors (OS) | VPA | Human fibroblasts | | 0.001% |
| 4 iPS factors (OSKM) | | Human keratinocytes | ~2 weeks | 1% |
| Adenoviral infection | | | | |
| 4 iPS factors (OSKM) | | Mouse hepatocytes | ~4 weeks | 0.006% |
| 4 iPS factors (OSKM) | | Mouse postnatal fibroblasts | | No colonies |
| Plasmid transfection | | | | |
| 4 iPS factors (OSKM) | | Mouse embryonic fibroblasts | | 0.0015% |
| Recombinant protein | | | | |
| 4 iPS factors (OSKM) | | Mouse embryonic fibroblasts | | No colonies |
| 4 iPS factors (OSKM) | VPA | Mouse embryonic fibroblasts | ~5 weeks | 0.006% |
| 3 iPS factors (OSK) | VPA | Mouse embryonic fibroblasts | | 0.002% |
| 4 iPS factors (OSKM) | | Human neonatal fibroblasts | ~8 weeks | 0.006% |

*O, Oct4; S, Sox2; K, Kif4; M, c-Myc
**VPA, Valproic acid

Application of current iPS cells in regenerative medicine is hampered by the use of viral delivery systems and tumor-related iPS factors such as c-myc and Klf4. In addition, the prospect for clinical application is impeded by the low efficiency of reprogramming and safety issues caused by viral vector transduction. Therefore, a need still exists for methods, compositions and kits that can be used to reprogram cells with improved efficiency and at the same time eliminate the dependence on viral vector-based systems.

### BRIEF SUMMARY

The invention relates to methods, compositions and kits for reprogramming a cell. Embodiments relate to methods comprising inducing the expression of a pluripotent or multipotent gene. In still yet another embodiment, the invention relates to a method comprising altering the activity of a protein that is involved in transcriptional repression. In yet another embodiment, the invention relates to a method for reprogramming a cell comprising altering the activity, expression or activity and expression of a regulatory protein. The method further comprises inducing the expression of a pluripotent or multipotent gene, and reprogramming the cell.

In still another embodiment, the invention relates to a method for improving the efficiency of reprogramming a cell. In yet another embodiment, the invention relates to a method for improving the efficiency of nuclear reprogramming. In still another embodiment, the invention relates to a method comprising reprogramming a cell using a purely chemical approach to induce reprogramming of a somatic nucleus to a less differentiated state.

In yet another embodiment, the invention relates to a method for reprogramming a cell comprising inducing an endogenous transcription factor. In still another embodiment, one or more than one transcription factor can be induced. In one embodiment, the method further comprises inducing the endogenous transcription factor network without the use of viral vectors and/or viral vector transduction.

In yet another embodiment, the invention relates to a method for reprogramming a cell comprising: delivering a transcription factor to a cell; contacting said cell with an agent that (1) reduces the activity, expression or activity and expression of a gene, which codes for a protein involved in transcriptional repression, and/or (2) reduces the activity of a protein involved in transcriptional repression; and selecting a cell, wherein differentiation potential has been restored to said cell. Delivery of a single transcription factor coupled with exposing the cell to an agent that reduces the activity, expression or activity and expression of a gene that codes for a protein, or reduces the activity of a protein involved in transcriptional repression is sufficient to efficiently and effectively reprogram a cell.

In one embodiment, delivering a transcription factor to a cell comprises delivering a transcription factor selected from the group consisting of Oct-4, Sox-2, klf4, Nanog, Lin28 and c-myc. In another embodiment, delivering a transcription factor to a cell comprises delivering Oct-4 and Sox-2. In still another embodiment, delivering a transcription factor to a cell comprises delivering no more than two transcription factors. In yet another embodiment, delivering a transcription factor to a cell comprises delivering no more than three transcription factors.

In another embodiment, methods for reprogramming a cell comprise (a) inducing expression of an endogenous transcription factor network within a cell with a first differentiation status; (b) selecting a cell with a second differentiation status; (c) expanding said selected cell into a population of cells. The second differentiation status has enhanced ability to differentiate into one or more than one cell type.

In yet another embodiment, methods for reprogramming a cell comprise (a) culturing a cell in a medium comprising an agent that reduces the activity, expression or activity and expression of a gene or protein involved in transcriptional repression; (b) delivering a transcription factor to said cell after culturing in said medium of (a); and (c) selecting a cell with increased differentiation potential.

In one embodiment, delivering said transcription factor comprises delivering the transcription factors Oct-4, Sox2, Klf4, and c-myc. In another embodiment, delivering said transcription factor comprises delivering transcription Oct-4, Sox-2, Nanog and Lin28.

In another embodiment, the invention relates to a method for reprogramming a cell comprising delivering a regulatory protein to a cell, inducing core somatic cell reprogramming factors, inducing the expression of a pluripotent or multipotent gene, and selecting a cell, wherein differentiation potential has been restored to said cell. Differentiation potential has been restored to a cell if the cell displays an increased ability to differentiate into one or more than one cell type.

In another embodiment, the regulatory protein is a transcription factor. In still another embodiment, the core somatic cell reprogramming factors are transcription factors. In yet another embodiment, the core somatic cell reprogramming factors are transcription factors that have a role in restoring differentiation potential to a cell including but not limited to Oct4, Sox2, Klf4, c-myc, Nanog, Dax1, Rex1, Zpf281, and Nac1.

In another embodiment, the method further comprises exposing said cell to an agent that (1) reduces the activity, expression or activity and expression of a gene that codes for a protein involved in transcriptional repression, and/or (2) reduces the activity of a protein involved in transcriptional repression.

Embodiments of the invention also relate to methods for reprogramming a cell comprising contacting a cell, a population of cells, a cell culture, a subset of cells from a cell culture, a homogeneous cell culture or a heterogeneous cell culture with an agent that induces expression of a transcription factor and exposing said cell to a second agent that reduces the activity, expression or activity and expression of a gene coding for a protein involved in transcriptional repression, inducing the expression of a pluripotent or multipotent gene, and selecting a cell, wherein differentiation potential has been restored to said cell. The method further comprises re-differentiating the cell. The agent inducing expression of a transcription factor can cause endogenous induction of a transcription factor or can supply the transcription factor that is to be induced, such as in the form of a replicable vector (plasmid, cosmid, virus, artificial chromosome).

In yet another embodiment, the invention relates to a method for reprogramming a cell comprising: delivering a transcription factor to a cell, exposing said cell to an agent that (1) reduces the activity, expression or activity and expression of a gene that codes for a protein involved in transcriptional repression, or (2) reduces the activity of a protein involved in transcriptional repression, inducing the expression of a pluripotent or multipotent gene, and selecting a cell, wherein differentiation potential has been restored to said cell. In yet another embodiment, the method further comprises inducing core somatic cell reprogramming factors through endogenous auto- and reciprocal transcriptional regulation.

A transcription factor or more than one transcription factor can be delivered to a cell of interest using any suitable method including but not limited to a lentivirus, a biological cassette, a plasmid, a yeast artificial chromosome, a small molecule, small molecule inhibitor, and a small molecule activator.

Embodiments of the invention also include methods for treating a variety of diseases using a reprogrammed cell produced according to the methods disclosed herein. In yet another embodiment, the invention also relates to therapeutic uses for reprogrammed cells and reprogrammed cells that have been re-differentiated.

Embodiments of the invention also relate to a reprogrammed cell produced by the methods of the invention. The reprogrammed cell can be re-differentiated into a single lineage or more than one lineage. The reprogrammed cell can be multipotent or pluripotent.

Embodiments of the invention also relate to kits for preparing the methods and compositions of the invention. The kit can be used for, among other things, reprogramming a cell and generating ES-like and stem cell-like cells.

An advantage of the methods disclosed herein is improved efficiency rates for reprogramming.

An advantage of the methods disclosed herein is faster reprogramming protocols.

### BRIEF DESCRIPTION OF THE DRAWINGS

FIG. 1 is a schematic of epigenetic modification and somatic cell reprogramming transcription factor network.

FIG. 2A is a photograph of various cell types infected with turboGFP lentiviral shRNA GAPDH. The cell types shown in FIG. 2A are: preadipocytes, human lung fibroblasts (HLF), skeletal muscle cells (SkM), and human dermal fibroblasts (HDF). Expression of turboGFP from lentiviral vector was visualized using fluorescent microscopy.

FIG. 2B is a bar graph reporting the level of GAPDH mRNA analyzed by real-time RT-PCR. Puromycin is abbreviated Puro.

FIG. 3A is a bar graph reporting the induction of Oct4 mRNA in HDF during shRNA-induced gene knockdown of DNMT1. Target gene expression (DNMT1, Oct4) was analyzed using real time qRT-PCR and normalized by GAPDH expression.

FIG. 3B are photographs depicting the expression of turboGFP, Oct4, Sox2 and SSEA4. Colony formation was visualized by immunocytochemistry using specific antibodies and green fluorescence.

FIG. 4A is a bar graph reporting induction of Nanog mRNA in HDF during shRNA-induced gene knockdown of HDAC7 (closed circle) and HDAC11 (open circle). Target gene expression was analyzed using real time qRT-PCR and normalized by GAPDH expression.

FIG. 4B is a bar graph reporting compensatory induction of HDAC mRNA by HDAC7 shRNA lentiviral infection. Target gene expression was analyzed using real time qRT-PCR and normalized by GAPDH expression.

FIG. 4C is a bar graph reporting HDAC and DNMT1 gene expression in BJFs (ATCC) treated with 500 nM Valproic acid (VPA) for 7 days. HDAC1 (P<0.01), SIRT4 (P<0.05) and DNMT1 (P<0.005) were significantly up regulated compared to control (MC) cells.

FIG. 5A is a bar graph reporting lentiviral overexpression of Oct4 and the effects on the expression of Oct-4 (closed circle) and Nanog (opened) in human dermal fibroblasts. Target gene expression was analyzed using real time qRT-PCR and normalized by GAPDH expression. The level of Oct4 expression in hES cells is about 1027.

FIG. 5B is a bar graph reporting lentiviral overexpression of Nanog and the effects on the expression of Oct-4 (closed circle) and Nanog (open circle) in human dermal fibroblasts. Target gene expression was analyzed using real time qRT-PCR and normalized by GAPDH expression. The level of Nanog expression in hES cells is about 932.

FIG. 5C are photographs depicting morphological changes by Oct4 and HDAC9 shRNA lentiviral infection. GFP positive cells were visualized under fluorescent microscope. hES cell was obtained from Invitrogen.

FIG. 6 is a schematic depicting a screening method for somatic cell reprogramming using the reprogramming transcription factor network.

FIG. 7 is a schematic depicting one representative cell culture timeline for activation of the endogenous transcription factor network. Representative small molecule inhibitors and representative concentrations are provided.

FIG. 8 is a panel of photographs of human adipose tissue derived stem cells (hADS) subjected to various treatments. FIG. 8A is a photograph of hADS cells transfected with Oct-4 (“O”), klf4 (“K”), Sox-2 (“S”, and c-myc (“M”). FIG. 8B is a photograph of hADS cells transfected with OSKM. FIGS. 8C and 8D are photographs of hADS cells pre-treated with the historic deacetylase inhibitor Scriptaid (“Scrip”). FIG. 8E and FIG. 8F are photographs of hADS cells pre-treated with the histone deacetylase inhibitor trichostatin A (TSA). FIG. 5G and FIG. 8H are hADS cells pre-treated with the histone deacetylase inhibitor valproic acid (VPA). Photographs were taken at 10× magnification.

FIG. 9 is a panel of photographs of human adipose tissue derived stem cells (hADS) subjected to various treatments. FIG. 9A and FIG. 9B are photographs of hADS cells transfected with Oct-4 (“O”), klf4 (“K”), Sox-2 (“S”, and c-myc (“M”). FIGS. 9C and 9D are photographs of hADS cells pre-treated with the histone deacetylase inhibitor trichostatin A (TSA). FIG. 9E and FIG. 9F are photographs of hADS cells pre-treated with the histone deacetylase inhibitor Scriptaid (“Scrip”). FIG. 9G and FIG. 9H are hADS cells pre-treated with the histone deacetylase inhibitor valproic acid (VPA). Photographs 9A, 9C, 9E, and 9G were taken at 10× magnification. Photographs 9B, 9D, 9F, and 9H were taken at 20× magnification.

FIG. 10 is a panel of photographs of human adipose tissue derived stem cells (hADS) demonstrating somatic cell reprogramming. Photographs A1, A2, A3, A4, A5, B1, B2, B3, B4, B5, C1, C2, C3, C4, C5, D1, D2, D3, D4, D5, E1, E2, and D3 are shown. Photographs B4, C4, C5, D2, and D3 depict cells displaying phenotypes of reprogrammed cells.

FIG. 11 is panel of photographs of human adipose tissue derived stem cells (hADS) treated with trichostatin A. FIGS. 11A, 11B, 11C, and 11D are photographs of hADS cells treated with TSA and treated with antibody that recognizes Sox-2. FIGS. 11E, 11F, 11G, and 11H are photographs of hADS cells treated with TSA and treated with antibody that recognizes Oct-4.

FIG. 12 is a panel of photographs of human adipose tissue derived stem cells (hADS). FIG. 12A-12D are photographs of hADS cells treated with antibody that recognizes Sox-2.

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, melt index, temperature etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

“Cell” or “cells,” unless specifically limited to the contrary, includes any somatic cell, embryonic stem (ES) cell, adult stem cell, an organ specific stem cell, nuclear transfer (NT) units, and stem-like cells. The cell or cells can be obtained from any organ or tissue. The cell or cells can be human or other animal. For example, a cell can be mouse, guinea pig, rat, cattle, horses, pigs, sheep, goats, etc. A cell also can be from non-human primates.

“Culture Medium” or “Growth Medium” refers to a suitable medium capable of supporting growth of cells.

“Delivering” refers to bringing an article to a second article, and is used interchangeably with the terms introducing, inserting, providing, and contacting.

“Differentiation” refers to the process by which cells become structurally and functionally specialized during embryonic development. Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type.

“DNA methyltransferase inhibitor” and “inhibitor of DNA methyltransferase” refer to a compound that is capable of interacting with a DNA methyltransferase and inhibiting its activity. “Inhibiting DNA methyltransferase activity” means reducing the ability of a DNA methyltransferase to methylate a particular substrate, such as a CpG dinucleotide sequence. In some embodiments, such reduction of DNA methyltransferase activity is at least about 25% at least about 50%, in other embodiments at least about 75%, and still in other embodiments at least about 90%. In yet another embodiment, DNA methyltransferase activity is reduced by at least 95% and in another embodiment by at least 99%.

“Endogenous transcription factor network” refers to a series of genes and proteins that contribute to maintaining the pluripotency state of a cell. Auto- and reciprocal-transcriptional regulation and protein-protein interactions contribute to maintaining appropriate levels of pluripotency gene expression in cells. The endogenous transcription factor network includes glycine N-methyltransferase (Gnmt), Octamer-4 (Oct4), Nanog, GABRB3, LEFTB, NR6A1, PODXL, PTEN, SRY (sex determining region Y)-box 2 (also known as Sox2), Sox-1, Sox-3, Sox-15, Myc, REX-1 (also known as Zfp-42), Integrin α-6, Rox-1, LIF-R, TDGF1 (CRIPTO), SALL4 (sal-like 4), Leukocyte cell derived chemotaxin 1 (LECT1), BUB1, FOXD3, NR5A2, TERT, LIFR, SFRP2, TFCP2L1, LIN28, XIST, Dax-1, Nac1, Zpf281, Esrrβ, Esrry, and Kruppel-like factors (Klf) such as Klf4 and Klf5.

“Epigenetics” refers to the state of DNA with respect to heritable changes in function without a change in the nucleotide sequence. Epigenetic changes can be caused by modification of the DNA, such as by methylation and demethylation, without any change in the nucleotide sequence of the DNA.

“Exposing” refers to placing a first article in the proximity of a second article. “Exposing” may result in the first article having direct or indirect contact with a second article. “Exposing” includes contacting, placing and touching.

“Expression construct” or “expression cassette” refers to a nucleic acid molecule that is capable of directing transcription. An expression construct includes, at the least, a promoter or a structure functionally equivalent to a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

“Histone” refers to a class of protein molecules found in chromosomes responsible for compacting DNA enough so that it will fit within a nucleus.

“Histone deacetylase inhibitor” and “inhibitor of histone deacetylase” refer to a compound that is capable of interacting with a histone deacetylase and inhibiting its enzymatic activity. “Inhibiting histone deacetylase activity” refers to reducing the ability of a histone deacetylase to remove an acetyl group from a suitable substrate, such as a histone, or other protein. In some embodiments, such reduction of histone deacetylase activity is at least about 50%, in other embodiments at least about 75%, and still in other embodiments at least about 90%. In still yet other embodiments, histone deacetylase activity is reduced by at least 95% and in other embodiments by at least 99%.

“Knock down” refers to suppressing the expression of a gene in a gene-specific fashion. A cell that has one or more genes “knocked down,” is referred to as a knock-down organism or simply a “knock-down.”

A “plasmid” refers to an extra-chromosomal DNA molecule separate from the chromosomal DNA that is capable of replicating independently of the chromosomal DNA. In certain cases, it is circular and double-stranded.

“Pluripotent” is capable of differentiating into cell types of the 3 germ layers or primary tissue types.

“Pluripotent gene” is a gene that contributes to a cell being pluripotent.

“Pluripotent cell cultures” are said to be “substantially undifferentiated” when they display morphology that clearly distinguishes them from differentiated cells of embryo or adult origin. Pluripotent cells typically have high nuclear/cytoplasmic ratios, prominent nucleoli, and compact colony formation with poorly discernable cell junctions, and can be ecognized by those skilled in the art. It is recognized that colonies of undifferentiated cells can be surrounded by neighboring cells that are differentiated. Nevertheless, the substantially undifferentiated colony will persist when cultured under appropriate conditions, and undifferentiated cells constitute a prominent proportion of cells growing upon splitting of the cultured cells. Useful cell populations described in this disclosure contain any proportion of substantially undifferentiated pluripotent cells having these criteria. Substantially undifferentiated cell cultures may contain at least about 20%, 40%, 60%, or even 80% undifferentiated pluripotent cells (in percentage of total cells in the population).

“Regulatory protein” is any protein that regulates a biological process, including regulation in a positive and negative direction. The regulatory protein can have direct or indirect effects on the biological process, and can either exert affects directly or through participation in a complex.

“Reprogramming” refers to removing epigenetic marks in the nucleus, followed by establishment of a different set of epigenetic marks. During development of multicellular organisms, different cells and tissues acquire different programs of gene expression. These distinct gene expression patterns appear to be substantially regulated by epigenetic modifications such as DNA methylation, histone modifications and other chromatin binding proteins. Thus each cell type within a multicellular organism has a unique epigenetic signature that is conventionally thought to become “fixed” and immutable once the cells differentiate or exit the cell cycle. However, some cells undergo major epigenetic “reprogramming” during normal development or certain disease situations

In addition, “reprogramming” is a process that confers on a cell a measurably increased capacity to form progeny of at least one new cell type, either in culture or in vivo, than it would have under the same conditions without reprogramming. More specifically, “reprogramming” is a process that confers on a somatic cell a pluripotent potential. This means that after sufficient proliferation, a measurable proportion of progeny having phenotypic characteristics of the new cell type if essentially no such progeny could form before reprogramming; otherwise, the proportion having characteristics of the new cell type is measurably more than before reprogramming. Under certain conditions, the proportion of progeny with characteristics of the new cell type may be at least about 1%, 5%, 25% or more in the in order of increasing preference.

“Small molecule modulator” refers to compounds that are small molecule inhibitors or small molecule activators. A small molecule modulator may function as a small molecule inhibitor in some physiological contexts and as a small molecule activator in another physiological context. A small molecule modulator may function as a small molecule inhibitor with regard to one target, and as a small molecule activator with regard to another target. The same small molecule modulator may function as both a small molecule activator and as a small molecule inhibitor.

“Totipotent” is capable of developing into a complete embryo or organism,

A “vector” or “construct” (sometimes referred to as gene delivery or gene transfer “vehicle”) refers to a macromolecule or complex of molecules comprising a polynucleotide to be delivered to a host cell, either in vitro or in vivo.

Embodiments of the invention relate to methods comprising inducing expression of at least one gene that contributes to a cell being pluripotent or multipotent. In some embodiments, the methods induce expression of at least one gene that contributes to a cell being pluripotent or multipotent and producing reprogrammed cells that are capable of directed differentiation into at least one lineage.

Embodiments of the invention also relate to a method comprising modifying chromatin structure, and reprogramming a cell to be pluripotent or multipotent. In yet another embodiment, modifying chromatin structure comprises delivering a transcription factor and exposing a cell to an agent that reduces the activity, expression or activity and expression of a gene, which codes for a protein, or a protein involved in transcriptional repression.

In yet another embodiment, the invention relates to a method for reprogramming a cell comprising: delivering a transcription factor to a cell; exposing said cell to an agent that reduces the activity, expression or activity and expression of a gene, which codes for a protein, or reduces the activity of a protein involved in transcriptional repression, inducing core somatic cell reprogramming factors through endogenous auto—and reciprocal transcriptional regulation, and inducing the expression of at least one gene that contributes to a cell being pluripotent or multipotent. In yet another embodiment, the method further comprises producing a reprogrammed cell.

In still another embodiment, the invention relates to a method for reprogramming a cell comprising: delivering a transcription factor to a cell, inducing core somatic cell reprogramming factors through endogenous auto- and reciprocal transcriptional regulation, and selecting a cell, wherein differentiation potential has been restored.

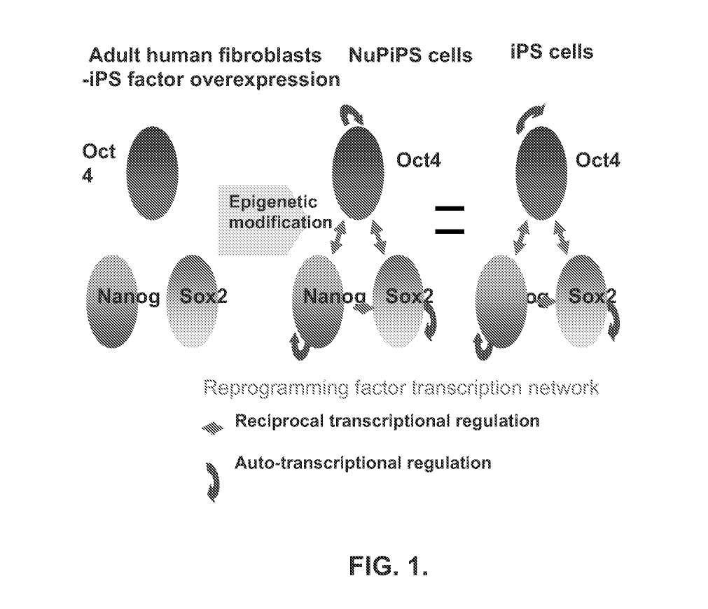

The endogenous somatic cell reprogramming factor transcription network is unique in embryonic stem cells, and is minimally active, if at all, in adult human dermal fibroblasts. The endogenous transcription factor network includes auto- and reciprocal-transcriptional regulation and protein-protein interactions that contribute to maintaining appropriate levels of pluripotency gene expression in embryonic stem cells (see FIG. 1). Nuclear reprogramming can be induced by epigenetic modification(s) that activate the endogenous transcription factor network in somatic cells as shown in the FIG. 1. Chromatin modification by silencing repressive epigenetic regulatory components (e.g., HDACs and/or DNMTs) in human dermal fibroblasts is likely to increase somatic cell reprogramming efficiency.

In one embodiment, the invention relates to a method for reprogramming a cell comprising: delivering a transcription factor to a cell; exposing said cell to an agent that reduces the activity, expression or activity and expression of a gene coding for a protein involved in transcriptional repression, inducing the expression of a pluripotent or multipotent gene, and selecting a cell, wherein differentiation potential has been restored to said cell. The pluripotent or multipotent gene may be induced by any fold increase in expression including but not limited to 0.25-0.5, 0.5-1, 1.0-2.5, 2.5-5, 5-10, 10-15, 15-20, 20-40, 40-50, 50-100, 100-200, 200-500, and greater than 500. In another embodiment, the method comprises plating differentiated cells, delivering a transcription factor to said plated cells; exposing said plated cells to an agent that reduces the activity, expression or activity and expression of a gene coding for a protein involved in transcriptional repression, culturing said cells, and identifying a cell with restored differentiation potential.

The activity or expression of a regulatory protein can be increased or decreased by any amount including but not limited to 1-5%, 5-10%, 10-20%, 20-30%, 30-40%, 40-50%, 50-60%, 60-70%, 70-80%, 80-90%, 90-95%, and 95-99%, 99-200%, 200-300%, 300-400%, 400-500% and greater than 500%.

In yet another embodiment, the method further comprises selecting a cell using an antibody directed to a protein or a fragment of a protein coded for by a pluripotent or multipotent gene or an antibody directed to a pluripotent or multipotent marker. Any type of antibody can be used including but not limited to a monoclonal, a polyclonal, a fragment of an antibody, a peptide mimetic, an antibody to the active region, and an antibody to the conserved region of a protein In still another embodiment, the method comprises selecting a cell and expanding or culturing said cell to a pluripotent cell culture.

In still another embodiment, the method further comprises selecting a cell using a reporter driven by a pluripotent or multipotent gene or a pluripotent or multipotent surface marker. Any type of reporter can be used including but not limited to a fluorescent protein, green fluorescent protein, cyan fluorescent protein (CFP), a yellow fluorescent protein (YFP), bacterial luciferase, jellyfish aequorin, enhanced green fluorescent protein, chloramphenicol acetyltransferase (CAT), dsRED, β-galactosidase, and alkaline phosphatase.

In still another embodiment, the method further comprises selecting a cell using resistance as a selectable marker including but not limited to resistance to an antibiotic, a fungicide, puromycin, hygromycin, dihydrofolate reductase, thymidine kinase, neomycin resistance (neo), G418 resistance, mycophenolic acid resistance (gpt), zeocin resistance protein and streptomycin.

In still another embodiment, the method further comprises comparing the chromatin structure of a pluripotent or multipotent gene of a cell that exists before delivering a transcription factor to said cell and exposing said cell to an agent that reduces the activity, expression or activity and expression of a gene coding for a protein involved in transcriptional repression to the chromatin structure of a pluripotent or multipotent gene obtained after delivering said transcription factor and exposure to said agent. Any aspect of chromatin structure can be compared including but not limited to euchromatin, heterochromatin, histone acetylation, histone methylation, the presence and absence of histone or histone components, the location of histones, the arrangement of histones, and the presence or absence of regulatory proteins associated with chromatin. The chromatin structure of any region of a gene may be compared including but not limited to an enhancer element, an activator element, a promoter, the TATA box, regions upstream of the start site of transcription, regions downstream of the start site of transcription, exons and introns.

In still another embodiment, the invention relates to a method comprising exposing a cell with a first phenotype to a first agent that induces expression of a transcription factor; exposing said cell to a second agent that reduces the activity, expression, or activity and expression of a gene, which codes for a protein, or a protein involved in transcriptional repression, comparing the first phenotype of the cell to a phenotype obtained after exposing the cell to said first and second agents, and selecting the cell with restored differentiation potential. In yet another embodiment, the method comprises comparing the genotype of a cell prior a exposing the cell to a first agent that induces expression of a transcription factor; and a second agent that reduces the activity, expression or activity and expression of a gene, which codes for a protein, or reduces the activity of a protein involved in transcriptional repression to a genotype of the cell obtained after exposure to said first and second agents.

In still yet another embodiment, the method comprises comparing the phenotype and genotype of a cell prior to exposing said cell to a first agent that induces expression of a transcription factor and a second agent that reduces the activity, expression, or activity and expression of a gene, which codes for a protein, or reduces the activity of a protein involved in transcriptional repression to the phenotype and genotype of the cell obtained after exposing the cell to said first and second agents.

In still another embodiment, the method comprises culturing or expanding the selected cell to a population of cells. In yet another embodiment, the method comprises isolating cells using an antibody that binds to a protein coded for by a pluripotent or multipotent gene or an antibody that binds to a multipotent marker or a pluripotent marker, including but not limited to SSEA3, SSEA4, Tra-1-60, and Tra-1-81, Oc-4, Sox-2, and Nanog. In still another embodiment, the invention further comprises comparing chromatin structure of a pluripotent or multipotent gene prior to exposure to a first agent that induces expression of a transcription factor; and a second agent that reduces the activity, expression, or activity and expression of a gene, which codes for a protein, or a protein involved in transcriptional repression to the chromatin structure obtained after exposure to said first and second agent. Cells also may be isolated using any method that is suitable and efficient for isolating cells including but not limited to a fluorescent cell activated sorter, immunohistochemistry, and ELISA. In another embodiment, the method comprises selecting a cell that has a less differentiated state than the original cell.

In another embodiment, the invention relates to a method for reprogramming a cell comprising: delivering a transcription factor to a cell with a first transcriptional pattern, exposing said cell to an agent that reduces the activity, expression, or activity and expression of a gene, which codes for a protein, or reduces the activity of a protein involved in transcriptional repression, comparing the first transcriptional pattern of the cell to a transcriptional pattern obtained after delivering said transcription factor and exposure to said agent; and selecting a cell, wherein differentiation potential has been restored to said cell. In another embodiment, selecting a cell comprises selecting a cell that has a less differentiated state than the original cell.

In still another embodiment, selecting a cell comprises identifying a cell with a transcriptional pattern that is at least 5-10%, 10-20%, 20-30%, 30-40%, 40-50%, 50-60%, 60-70%, 70-80%, 80-90%, 90-94%, 95%, or 95-99% similar to an analyzed transcriptional pattern of an embryonic stem cell. The entire transcriptional pattern of an embryonic stem cell need not be compared, although it may. Instead, a subset of embryonic genes may be compared including but not limited to 1-5,5-10, 10-25, 25-50, 50-100, 100-200, 200-500, 500-1,000, 1,000-2,000, 2,000-2,500, 2,500-5,000, 5,000-10,000 and greater than 10,000 genes. The transcriptional patterns may be compared in a binary fashion, i.e., the comparison is made to determine if the gene is transcribed or not. In another embodiment, the rate and/or extent of transcription for each gene or a subset of genes may be compared. Transcriptional patterns can be determined using any methods known in the art including but not limited to RT-PCR, quantitative PCR, a microarray, southern blot and hybridization.

In yet another embodiment, the invention relates to a method for reprogramming a cell comprising: delivering a transcription factor to a cell; exposing said cell to an agent that reduces the activity, expression, or activity and expression of a gene, which codes for a protein, or reduces the activity of a protein involved in transcriptional repression; and monitoring the efficiency of reprogramming. In another embodiment, the efficiency of reprogramming is based on the percentage of reprogrammed cells. The methods of invention can be used to achieve high efficiency of reprogramming including but not limited to 1, 2, 3, 4, 5, 6, 7, 8, 9, 10, 11, 12, 13, 14, 15, 16, 17, 18, 19, 20, 21, 22, 23, 24, 25, 25-30, 30-35, 35-40, 40-45, 45-50, 50-55, 55-60, 60-65, 65-70, 70-75, 75-80, 80-85, 85-90, 91, 92, 93, 94, 95, 96, 97, 98, 99, and greater than 99% reprogrammed cells.

In yet another embodiment, the invention relates to an enriched population of reprogrammed cells produced according to a method comprising delivering a transcription factor to a cell; exposing said cell to an agent that reduces the activity, expression, or activity and expression of a gene coding for a protein involved in transcriptional repression; inducing expression of a pluripotent or multipotent gene, selecting a cell, wherein differentiation potential has been restored to said cell, and culturing said selected cell to produce a population of cells. In still another embodiment, the reprogrammed cell expresses a cell surface marker selected from the group consisting of SSEA3, SSEA4, Tra-1-60, and Tra-1-81, Oct-4, Nanog, and Sox-2. In yet another embodiment, the reprogrammed cells account for at least 1-5%, 5-10%, 10-20%, 20-30%, 30-40%, 40-50%, 50-60%, 60-70%, 70-80%, 80-90%, 90-95%, 96-98%; or at least 99% of the enriched population of cells.

Agents that Reduce the Activity of a Gene or a Protein Involved in Transcriptional Repression

In one embodiment, cells can be treated with an agent that seduces the activity, expression, or activity and expression of a gene, which codes for a protein involved in transcriptional repression, or an agent that reduces the activity of a protein involved in transcriptional repression including but not limited to a RNA, shRNA, siRNA, an HDAC modulator, an HDAC inhibitor, an HDAC activator, a small molecule, a small molecule inhibitor, a small molecule activator and a small molecule modulator.

shRNA

An agent that impedes the expression of a gene that codes for a protein involved in transcriptional repression includes but is not limited to an shRNA molecule, an shRNAmir molecule, a combination of shRNA molecules, a combination of shRNAmir molecules, and a combination of shRNA and shRNAmir molecules.

Inhibiting the expression of a gene that codes for a protein involved in transcriptional repression can be accomplished by any appropriate mechanism including but not limited to RNA interference (RNAi). RNAi regulates gene expression via a ubiquitous mechanism by degradation of target mRNA in a sequence-specific manner. Small interfering RNA strands (siRNA) are key to the RNAi process, and have complementary nucleotide sequences to the targeted RNA strand. Specific RNAi pathway proteins are guided by the siRNA to the targeted messenger RNA (mRNA), where they “cleave” the target, breaking it down into smaller portions that can no longer be translated into protein.

shRNA is a sequence of RNA that makes a tight hairpin turn that can be used to silence gene expression; the use of shRNA is one approach to achieve RNA interference. In some embodiments, shRNA can be incorporated into a vector with a promoter, including but not limited to a U6 promoter, to ensure that the shRNA is expressed. The vector is usually passed on to daughter cells, allowing the gene silencing to be inherited. The shRNA hairpin structure is cleaved by the cellular machinery into short interfering RNA, which then is bound to the RNA-induced silencing complex (RISC). This complex binds to and cleaves mRNAs that match the siRNA that is bound to it.

The shRNA can be incorporated into a lentiviral construct. Lentivirus is a genus of slow viruses of the Retroviridae family, characterized by a long incubation period. Lentiviruses can deliver a significant amount of genetic information into the DNA of the host cell, and thus, are an efficient method of a gene delivery vector.

In another embodiment, an shRNA library can be used with the methods of the invention to identify factors involved in transcriptional repression, factors involved in chromatin remodeling, and factors that contribute to a cell being pluripotent or multipotent. In still another embodiment, the shRNA library can be from the The RNAi Consortium (TRC), which is a collaborative group of 11 world-renowned academic and corporate life science research groups whose mission is to create comprehensive tools for functional genomics research and make them broadly available to scientists worldwide. The TRC collection, developed at the Broad Institute of MIT and Harvard, currently consists of 159,000 pre-cloned shRNA constructs targeting 16,000 annotated human genes.

shRNA constructs, libraries and vectors can be custom made or can be purchased from commercial sources including but not limited to SMARTvector shRNA Lentiviral technology available from Dharmacon RNAi technologies (Thermo Scientific, Lafayette, Colo.), MISSION™ TRC shRNA, which is available from Sigma Aldrich (St. Louis, Mo.), TRC lentiviral shRNA library, which is available from Open Biosystems (Huntsville, Ala.), BLOCK-iT™ RNAi vectors that feature constitutive or inducible promoters, different selection markers, and viral delivery options, available with Lentiviral and Adenoviral vectors (Invitrogen, Carlsbad, Calif.).

Further, shRNA molecules directed toward specific targets also are available from commercial sources such as OriGene (Rockville, Md.), and Santa Cruz Biotechnology (Santa Cruz, Calif.). An inducible shRNA also is available from Clonetech (Mountainview, Calif.). The Knockout Inducible RNAi Systems tightly regulates the expression of functional short hairpin RNAs (shRNAs) in mammalian cells for the purpose of silencing target genes. Knockout Inducible RNAi Systems are useful in cases where suppression of a gene may be lethal, preventing its analysis. There are several versions available: The Knockout Single Vector Inducible RNAi system and The Knockout Tet RNAi Systems H and P.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to down-regulate gene expression.

Small Molecule Inhibitors

A small molecule inhibitor or “small molecular compound” refers to a compound useful in the methods, compositions, and kits of the invention having measurable or inhibiting activity. In some embodiments, the small molecule inhibitors have a relative molecular weight of not more than 1000 D, and in still other embodiments, of not more than 500 D. The small molecule inhibitor can be of organic or inorganic nature. In addition to small organic and inorganic compounds, peptides, antibodies, cyclic peptides and peptidomimetics are contemplated as being useful in the disclosed methods.

Small molecule inhibitors can be used to inhibit any protein involved in transcriptional repression including but not limited to histone deacetylases (HDAC), methyl binding domain proteins (MBD), methyl adenosyltransferases (MAT), DNA methyltransferases (DNMT), histone methyltransferase, and methyl cycle enzymes. In still yet another embodiment, more than one agent can be used to inhibit the activity of more than one protein involved in transcriptional repression including but not limited to a DNA methyltransferase, a histone deacetylase, a methyl binding domain protein, or a histone methyltransferase.

Preferably, such inhibition is specific, i.e., for a DNMT small molecule inhibitor, the DNMT inhibitor reduces the ability of a DNA methyltransferase to methylate a particular substrate or reduces the ability of a DNA methyltransferase to interact with another component required for methylation, at a concentration that is lower than the concentration of the inhibitor that is required to produce another, unrelated biological effect. Preferably, the concentration of the inhibitor required for DNA methyltransferase inhibitory activity is at least 2-fold lower, more preferably at least 5-fold lower, even more preferably at least 10-fold lower, and most preferably at least 20-fold lower than the concentration required to produce an unrelated biological effect.

Any number, any combination and any concentration of small molecule modulators can be used to alter the activity, expression, or activity and expression of a protein or more than one protein involved in transcriptional regulation including but not limited to 1, 2, 3, 4, 5, 6, 7, 8, 9, 10, 11-15, 16-20, 21-25, 25-50, 50-100, 100-250, and greater than 250. The small molecule modulator can be directed toward a specific protein or more than one protein, a specific class of proteins or more than one class of proteins, a specific family of proteins or more than one family of proteins or general transcriptional components.

A small molecule modulator may have an irreversible mechanism of action or a reversible mechanism of action. A small molecule modulator can have any binding affinity including but not limited to millimolar (mM), micromolar (μM), nanomolar (nM), picomolar (pM), and fentamolar (fM). A small molecule modulator can bind to a regulatory region or a catalytic region of the protein.

A DNMT small molecule inhibitor may interact with and inhibit any DNA methyltransferase including but not limited to DNMT1, DNMT2, DNMT3A, and DNMT3B, and DNMT3L. DNMT1 is likely the most abundant DNA methyltransferase in mammalian cells, and considered to be involved in maintenance methyltransferase in mammals. A DNMT inhibitor may interact with one type of DNMT, all types of DNMTs or with multiple types of DNMTs. A DNMT inhibitor of the invention may also interact with a DNMT that does not fall into one of the known types or is of yet unclassified.

Table I provides a representative list of small molecule inhibitors that can inhibit a DNMT. A DNMT inhibitor used in the methods, compositions, and kits of the invention include derivatives and analogues of a DNMT inhibitor herein mentioned.

TABLE I
Representative Examples of Nucleoside Analogues and
Non-Nucleoside Analogues that are DNMT Inhibitors

| Nucleoside Analogues | Non-Nucleoside Analogues |
|---|---|
| 5-Azacytidine | Hydralazin |
| 5-aza-2-deoxycytidine | Hydralazine Hydrochloride |
| 5-Fluoro-2-doecytodine | Procainamide |
| 5,6-dihydro-5-azacytidine, | Procaine |
| 5-fluroouracil | Procaine Hydrochloride |
| Zebularine | Epigallocatechin-3-gallate |
| | (EFOG) |
| | Psammaplin A |
| | MG98 |
| | RG108 |

Small Molecule Modulators

A small molecule modulator may be any of the compounds contained in a small molecule library or a modified compound derived from a compound contained in small molecule libraries. Several small molecule libraries are available from commercial sources including but not limited to BIOMOL INTERNATIONAL (now Enzo Life Sciences), and include but are not limited to Bioactive Lipid Library, Epi-drug library, Endocannabinoid Library, Fatty acid library, ICCB Known Bioactives Library, Ion Channel Ligand Library, Kinase Inhibitor Library, Kinase/Phosphatase Inhibitor Library, Neurotransmitter Library, Natural Products Library, Nuclear Receptor Library, Orphan Ligand Library, Protease Inhibitor Library, Phosphatase Inhibitor Library, and Rare Natural Products Library.

Table II is a representative list of small molecule modulators that can be used to induce, up-regulate or alter the expression of a gene involved in reprogramming. The small molecule modulator may target a component of the basal transcriptional machinery, a component of transcriptional activation, a component of a chromatin remodeling complex, a component of transcriptional repression, a component of DNA repair, a component of mismatch repair, and a component involved in maintaining the methylation state of a cell, a component of the Wnt-signaling pathway and a cyclin dependent kinase. Small molecule modulators include but are not limited to a histone deacetylase inhibitor (HDACi,), a histone acetyltransferase inhibitor (HATi), a histone acetyltransferase activator, a lysine methyltransferase inhibitor (LMTi,), a histone methyltransferase inhibitor (HMTi,), a Trichostatin A inhibitor (TSAi,), a histone demethylase inhibitor (HdeMi,), a lysine demethylase inhibitor (LdeMi), a sirtuin inhibitor (SIRTi,), and a sirtuin activator (SIRTa,).

TABLE II
Representative list of small molecule modulators
that can be used to reprogram a cell

| Small Molecule Modulator | Function |
|---|---|
| HC Toxin | HDACi |
| Garcinol | HATi |
| BML-210 | HDACi |
| Chaetocin | LMTi/HMTi |
| ITSA1 | TSAi |
| Depudecin | HDACi |
| Tranylcypromine | HdeMi/LdeMi |
| EX-527 | SIRT1i |
| Resveratrol | SIRT1a |
| M-344 | HDACi |
| Nicotinamide | SIRTi |
| Fluoro-SAHA | HDACi |
| Piceatanol | SIRTa |
| BML-266 | SIRT2i |
| Sirtinol | SIRT2i |
| Valproxam | HDACi |
| AGK2 | SIRT2i |
| CDKi | Cyclin dependent kinase |

Any small molecule modulator that functions as a histone acetyltransferase inhibitor can be used including but not limited to anacardic acid, garcinol, curcumin, isothiazolones, butyrolactone, and MC1626 (2-methyl-3-carbethoxyquinoline), polyisoprenylated Benzophenone, epigallocatechin-3-gallate (EGCG), and CPTH2 (cyclopentylidene-[4-(4′-chlorophenyl)thiazol-2-yl)hydrazone).

Any small molecule modulator that functions as a histone demethylase inhibitor can be used including but not limited to lysine specific demethylase, LSD1 (KIAA0601 or BHC110), flavin-dependent amine oxidase, and jumonji.

Any small molecule modulator that functions as a sirtuin activator can be used including but not limited to resveratrol, a polyphenol, a sirtuin activating compound, activators of SIRT1-SJRT7, and SRT-1720.

HDAC Inhibitors

An HDAC inhibitor of the methods, compositions and kits of the invention may interact with any HDAC. An HDAC inhibitor of the invention may interact with an HDAC of class I, class II, classIII, or class IV. An HDAC inhibitor may interact with one specific class of HDACs, all classes of HDACS, or with multiple classes of HDACs. An HDAC inhibitor may also interact with HDACs that do not fall into one of the known classes.

An HDAC inhibitor may have an irreversible mechanism of action or a reversible mechanism of action. An HDAC inhibitor can have any binding affinity including but not limited to millimolar (mM), micromolar (μM), nanomolar (nM), picomolar (pM), and fentamolar (fM).

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

In another embodiment, the HDAC inhibitor may act by binding to the zinc containing catalytic domain of the HDACs. HDAC inhibitors with this mechanism of action fall into several groupings: (i) hyroxamic acids, such as Trichostatin A; (ii) cyclic tetrapeptides; (iii) benzamides; (iv) electrophilic ketones; and (v) the aliphatic acid group of compounds such as phenylbutyrate and valproic acid.

In yet another embodiment, the FIDAC inhibitor can be directed toward the sirtuin Class III HDACs, which are NAD+ dependent and include but are not limited to nicotinamide, derivatives of NAD, dihydrocoumarin, naphthopyranone, and 2-hydroxynaphaldehydes.

In yet another embodiment, the HDAC inhibitor can alter the degree of acetylation of nonhistone effector molecules and thereby increase the transcription of genes. HDAC inhibitors of the methods, compositions, and kits of the invention should not be considered to act solely as enzyme inhibitors of HDACs. A large variety of nonhistone transcription factors and transcriptional co-regulators are known to be modified by acetylation, including but not limited to ACTR, cMyb, p300, CBP, E2F1, EKLF, FEN 1, GATA, HSP90, Ku70, NFκB, PCNA, p53, RB, Runx, SF1 Sp3, STAT, TFIIE, TCF, and YY1. The activity of any transcription factor or protein involved in activating transcription, which is acetylated, could be increased with the methods of the invention.

Table III provides a representative list of compounds that can function as an HDAC inhibitor. The reference to “Isotype” in Table V is meant to merely provide insight as to whether the compound has a preference for a particular class of HDAC. Listing a specific isotype or class of HDAC should not be construed to mean that the compound only has affinity for that isotype or class. HDAC inhibitors of the present invention include derivatives and analogues of any HDAC inhibitor herein mentioned.

Butyric acid, or butyrate, was the first HDAC inhibitor to be identified. However, in millimolar concentrations, butyrate may not be specific for HDAC, it also may inhibit phosphorylation and methylation of nucleoproteins as well as DNA methylation. The analogue, phenylbutyrate, acts in a similar manner. More specific are trichostatin A (TSA) and trapoxin (TPX). TPX and TSA have emerged as inhibitors of histone deacetylases. TSA reversibly inhibits, whereas TPX irreversibly binds to and inactivates HDAC enzymes. Unlike butyrate, nonspecific inhibition of other enzyme systems has not yet been reported for TSA or TPX.

Valproic acid (VPA) also inhibits histone deacetylase activity. VPA is a known drug with multiple biological activities that depend on different molecular mechanisms of action. VPA is an antiepileptic drug. VPA is teratogenic. When used as antiepileptic drug during pregnancy, VPA may induce birth defects (neural tube closure defects and other malformations) in a few percent of born children. In mice, VPA is teratogenic in the majority of mouse embryos when properly dosed. VPA activates a nuclear hormone receptor (PPAR-delta.).

TABLE III
A representative list of compounds that
can function as an HDAC inhibitor.

| | | Affinity | |
|---|---|---|---|
| HDAC Inhibitors | Isotype | Range | Chemical Class |
| Butyrate/Sodium Butyrate | class I/IIa | mM | carboxylate |
| Phenyl Butyrate | | | carboxylate |
| Valproic acid (VPA) | class I/IIa | mM | carboxylate |
| AN-9, Pivaloyloxymethyl | n/a | uM | carboxylate |
| butyrate | | | |
| m-Carboxycinnamic acid | n/a | uM | hydroxamate |
| bishydroxamic acid (CBHA) | | | |
| ABHA (azeleic | n/a | uM | hydroxamate |
| bishydroxamic acid) | | | |
| Oxamflatin | n/a | uM | hydroxamate |
| HDAC-42 | | | hydroxamate |
| SK-7041 | HDAC1/2 | nM | hydroxamate |
| DAC60 | | | hydroxamate |
| UHBAs | | | |
| Tubacin | HDAC6 | | hydroxamate |
| Trapoxin B | | | cyclic |
| | | | peptide/epoxide |
| A-161906 | n/a | | hydroxamate |
| R306465/JNJ16241199 | HDAC1/8 | | hydroxamate |
| SBHA (suberic | n/a | uM | hydroxamate |
| bishydroxamate) | | | |
| 3-CI-UCHA | | | |
| ITF2357 | class I/II | nM | hydroxamate |
| PDX-101 | class I/II | uM | hydroxamate |
| Pyroxamide | class I, | uM | hydroxamate |
| | unknown | | |
| | class II | | |
| Scriptaid | n/a | uM | hydroxamate |
| Suberoylanilide hydroxamic | class I/II/IV | uM | hydroxamate |
| acid)/Vorinostat/Zolinza | | | |
| Trichostatin A (TSA) | class I/II | nM | hydroxamate |
| LBH-589 (panobinostat) | class I/II | nM | hydroxamate |
| NVP-LAQ824 | class I/II | nM | hydroxamate |
| Apicidin | HDAC 2/3 | nM | cyclic peptide |
| Depsipeptide/FK- | class I/II | | peptide |
| 228/Romidepsin/FR901228 | | | |
| TPX-HA analogue (CHAP); | | nM | hydroxamate |
| CHAP1, CHAP31, CHAP50 | | | |
| CI-994(N-acetyl dinaline) | HDAC 1/2 | nM | benzamide |
| MS-275 (same as MS-27- | HDAC 1 | nM | benzamide |
| 275) | | | |
| PCK-101 | | | |
| MGCD0103 | HDAC 1/2 | nM | benzamide |
| Diallyl disulfide (DADS) | n/a | uM | disulfide |
| Sulforaphane (SFN) | n/a | uM | isothiocyanate |
| Sulforaphene (SFN with a | n/a | uM | isothiocyanate |
| double bond) | | | |
| Erucin | n/a | n/a | isothiocyanate |
| Phenylbutyl isothiocyanate | n/a | uM | isothiocyanate |
| Retinoids | | | |
| SFN-N-acetylcysteine (SFN- | n/a | uM | isothiocyanate |
| NAC) | | | |
| SFN-cysteine (SFN-Cys) | n/a | uM | isothiocyanate |
| Biotin | n/a | n/a | methyl-acceptor |
| Alpha-lipoic acid | n/a | n/a | carboxylate |
| Vit E metabolites | n/a | n/a | |
| Trifluoromethyl ketones | useful | nM | trifluoromethyl |
| | | | ketones |
| Alpha-Ketoamides | | | |
| splitomicin | class III | | |
| LAQ824 | class I/II | nM | hydroxamate |
| SK-7068 | HDAC1/2 | nM | hydroxamate |
| Panobinostat | class I/II | nM | hydroxamate |
| Belinostat | class I/II | nM | hydroxamate |

A variety of HDAC inhibitors also are available from Sigma Aldrich (St. Louis, Mo.) including but not limited to APHA Compound; Apicidin; Depudecin; Scriptaid; Sirtinol; and Trichostatin A. Further, additional HDAC inhibitors are available from Vinci-Biochem (Italy) including but not limited to 5-Aza-2′-deoxycytidine; CAY10398; CAY10433; 6-Chloro-2,3,4,9-tetrahydro-1H-carbazole-1-carboxamide; HC Toxin; ITSA1; M344; MC 1293; MS-275; Oxamflatin; PXD101; SAHA; Scriptaid; Sirtinol; Splitomicin. Dexamethasone may also be used in combination with any HDAC inhibitor. For example, a composition comprising dexamethasone and to 5-Aza-2′-deoxycytidine can be used.

Any number, any combination and any concentration of HDAC inhibitors can be used, including but not limited to 1, 2, 3, 4, 5, 6, 7, 8, 9, 10, 11-15, 16-20, and 21-25 HDAC inhibitors. One or more than one family of inhibitory proteins may be inhibited. One or more than one mechanism of inhibition may be used including but not limited to small molecule inhibitors, MAC inhibitors, shRNA, RNA interference, and small interfering RNA.

Genes and Proteins Involved in Transcriptional Repression

Any gene, which codes for a protein, or any protein involved in transcriptional repression can be inhibited by the methods of the invention including but not limited to DNA methyltransferases, histone deacetylases, methyl binding domain proteins, histone methyltransferases, a component of a chromatin remodeling complex, a component of the SWI/SNF complex, a component of the NuRD complex, and a components of the INO80 complex. A single gene or more than one gene can be inhibited by the methods of the invention including but not limited to 2, 3, 4, 5, 6, 7, 8, 9, 10, 11-20, 21-30, 31-40, 41-50, and greater than 50 genes.

A representative list of proteins that may be inhibited by the methods of the invention is provided in Table IV.

TABLE IV
Representative list of Proteins involved in transcriptional repression

| | Methyl | Meth. | DNA | Histone | |
|---|---|---|---|---|---|
| | Binding | Adenosyl- | Methyl- | Methyl- | Methyl |
| Histone | Domain | transfer- | transfer- | transfer- | Cycle |
| Deacetylases | Proteins | ases | ases | ase | Enzymes |
| Class 1 | MBD1 | MAT2A | DNMT1 | EHMT1 | MTHFR |
| (HDACs 1- | | | | | |
| 3, 8, 11 | | | | | |
| Class II | MBD2 | MAT1A | DNMT2 | HDM | CBS |
| (HDACs 4- | | | | G9A | |
| 7, 9, 10) | | | | | |
| Class III | MBD3 | MAT2B | DNMT3A | SUV39H1 | |
| (SIRTI 1-7) | | | | | |
| Class IV | MBD4 | | DNMT3B | SETDB1 | |
| (HDAC 11) | | | | | |
| | MeCP2 | | DNMT3L | | |

Any component of the Sin3 complex can be inhibited by the methods of the invention including but not limited to HDAC1, HDAC2, RbAp46, RbAp48, Sin3A, SAP30, and SAP18.

Any component of the NuRD complex can be inhibited by the methods of the invention including but not limited to Mi2, p70, and p32.

Any component of the 1N080 complex can be inhibited by the methods of the invention including but not limited to Tip49A, Tip49B, the SNF2 family helicase Ino80, actin related proteins ARK ARP5, and Arp8, YEATS domain family member Taf14, HMG-domain protein, Nhp10, and six additional proteins designated Ies1-6.

In one embodiment, the activity of polycomb group proteins can be modulated. Stems cells rely on Polycomb group proteins (PcG) to reversibly repress genes encoding transcription factors required for differentiation (Ringrose & Paro, Annu Rev Genet. 38:413-443, 2004). Lee et al. have identified genes targeted for transcriptional repression in human embryonic stem (ES) cells by the PcG proteins SUZ12 and EED, which form the Polycomb Repressive Complex 2, PRC2, and which are associated with nucleosomes that are trimethylated at histone H3 lysine-27 (H3K27me3) (Lee, T. I. et al. Cell 125:301-313, 2006, incorporated herein by reference, including supplemental materials thereof).

Reprogramming Factors

The following factors or combination thereof could be used in the methods disclosed herein. In one embodiment, nucleic acids encoding Sox and Oct (preferably Oct3/4) will be included into the reprogramming vector. For example, a reprogramming vector may comprise expression cassettes encoding Sox-2, Oct-4, Nanog and optionally Lin-28, or expression cassettes encoding Sox-2, Oct-4, Klf4 and optionally c-myc, or expression cassettes encoding Sox-2, Oct-4, and optionally Esrrb. Nucleic acids encoding these reprogramming factors may be comprised in the same expression cassette, different expression cassettes, the same reprogramming vector, or different reprogramming vectors.

Any gene and associated family members of that gene, which contribute to a cell being pluripotent or multipotent, may be induced, over-expressed or delivered to a cell of interest by the methods of the invention including but not limited to glycine N-methyltransferase (Gnmt), Octamer-4 (Oct4), Nanog, GABRB3, LEFTB, NR6A1, PODXL, PTEN, SRY (sex determining region Y)-box 2 (also known as Sox2), Myc, REX-1 (also known as Zfp-42), Integrin α-6, Rox-1, LIF-R, TDGF1 (CRIPTO), SALL4 (sal-like 4), Leukocyte cell derived chemotaxin 1 (LECT1), BUB1, FOXD3, NR5A2, TERT, LIFR, SFRP2, TFCP2L1, LIN28, XIST, Dax-1, Nac1, Zpf281, Esrrβ, Esrrγ, and Krüppel-like factors (Klf) such as Klf4 and Klf5. Any number of genes that contribute to a cell being pluripotent or multipotent can be induced or overexpressed by the methods of the invention including but not limited to 1, 2, 3, 4, 5, 6, 7, 8, 9, 10, 11-20, 21-30, 31-40, 41-50, and greater than 50 genes.

Oct-3/4 and certain members of the Sox gene family (Sox-1, Sox-2, Sox-3, and Sox-15) have been identified as crucial transcriptional regulators involved in the induction process whose absence makes induction impossible. Additional genes, however, including certain members of the Klf family (Klf-1, Klf2, Klf4, and Klf5), the Myc family (C-myc, L-myc, and N-myc), Nanog, and LIN28, have been identified to increase the induction efficiency.

Further, Ramalho-Santos et al. (Science 298, 597 (2002), Ivanova et al. (Science 298, 601 (2002) and Fortunel et al. (Science 302, 393b (2003)) (all incorporated by reference in their entirety) each compared three types of stem cells and identified a list of commonly expressed “sternness” genes, proposed to be important for conferring the functional characteristics of stem cells. Any of the genes identified in the above-mentioned studies may be induced by the methods of the invention. Table V provides a list of genes thought to be involved in conferring the functional characteristics of stem cells. In addition to the genes listed in Table V, 93 expressed sequence tags (EST) clusters with little or no homology to known genes were also identified by Ramalho-Santos et al. and Ivanova et al, and are included within the methods of the invention.

TABLE V
Genes implicated in conferring stem cell characteristics

| symbol | Gene | Function |
|---|---|---|
| F2r | Thrombin receptor | G-protein coupled receptor, |
| | | coagulation cascade, required for |
| | | vascular development |
| Ghr | Growth hormone | Growth hormone receptor/ |
| | receptor | binding protein, activates |
| | | Jak2 |
| Itga6 | Integrin alpha 6 | cell adhesion, cell-surface |
| | | mediated signalling, can |
| | | combine with Integrin b1 |
| Itgb1 | Integrin beta 1 | cell adhesion, cell-surface |
| | (fibronectin Receptor) | mediated signalling, can |
| | | combine with Integrin a6 |
| Adam 9 | A disintegrin and | cell adhesion, extracellular |
| | metalloproteinase | proteolysis, possible |
| | domain 9 (meltrin | fusogenic function |
| | gamma) | |
| Bys | Bystin-like (Bystin) | cell adhesion, may be |
| | | important for embryo implan- |
| | | tation (placenta) |
| Ryk | Receptor-like tyrosine | unconventional receptor |
| | kinase | tyrosine kinase |
| Pkd2 | Polycystic kidney | calcium channel |
| | disease 2 | |
| Kcnab3 | Potassium voltage | Regulatory subunit of potassium |
| | gated channel, shaker | channel |
| | related subfamily, beta | |
| | member 3 | |
| Gnb1 | Guanine nucleotide | G-protein coupled receptor |
| | binding protein beta 1 | signaling |
| Gab1 | Growth factor receptor | integration of multiple |
| | bound protein 2 | signaling pathways |
| | (Grb2) - associated | |
| | protein 1 | |
| Kras2 | Kirsten rat sarcoma | binds GTP and transmits signals |
| | oncogene 2 | from growth factor receptors |
| | ESTs highly similar to | suppressor of RAS function |
| | Ras p21 protein | |
| | activator (Gap) | |
| Cttn | Cortactin | regulates actin cytoskeleton, |
| | | overexpressed in tumors |
| Cops4 | COP9 (constitutive | Cop9 signalosome, integration |
| | photomorphogenic), | of multiple signaling path- |
| | subunit 4 | ways, regulation of protein |
| | | degradation |
| Cops7a | COP9 (constitutive | Cop9 signalosome, integration |
| | photomorphogenic), | of multiple signaling path- |
| | subunit 7a | ways, regulation of protein |
| | | degradation |
| Madh1 | Mad homolog 1 | TGFb pathway signal transducer |
| | (Smad1) | |
| Madh2 | Mad homolog 2 | TGFb pathway signal transducer |
| | (Smad2) | |
| Tbrg1 | TGFb regulated 1 | induced by TGFb |
| Stam | signal transducing | Associates with Jak tyrosine |
| | adaptor molecule (SH3 | kinase |
| | domain and ITAM | |
| | motif) 1 | |
| Statip1 | STAT interacting | scaffold for Jak/Stat3 binding |
| | protein 1 | |
| Cish2 | Cytokine inducible | STAT induced STAT inhibitor- |
| | SH2-containing | 2, interacts with Igf1R |
| | protein 2 (Ssi2) | |
| | ESTs moderately | possible tyrosine kinase |
| | similar to Jak3 | |
| | ESTs highly similar to | regulatory subunit of protein |
| | PPP2R1B | phosphatase 2, putative tumor |
| | | suppressor |
| Rock2 | Rho-associated coiled- | serine/theonine kinase, target |
| | coil forming kinase 2 | of Rho |
| Yes | Yamaguchi sarcoma | intracellular tyrosine kinase, |
| | viral oncogene | proto-oncogene, Src family |
| | homolog | |
| Yap | Yes-associated pro- | bind Yes, transcriptional co- |
| | tein 1 | activator |
| Ptpn2 | Protein tyrosine non- | dephosphorylates proteins |
| | receptor phosphatase 2 | |
| Ppplr2 | Protein phosphatase 1, | Inhibitory subunit of protein |
| | regulatory (inhibitor) 2 | phosphatase 1 |
| Ywhab | Tyrosine/tryptophan | Binds phosphoserine-proteins, |
| | monooxgenase | PKC pathway |
| | activation protein beta | |
| | (14-3-3beta) | |
| Ywhah | Tyrosine/tryptophan | Binds phosphoserine-proteins, |
| | monooxgenase | PKC pathway |
| | activation protein eta | |
| | (14-3-3eta) | |
| Axo | Axotrophin | contains a PHD domain, an |
| | | adenylaye cyclase domain and a |
| | | consensus region for G-protein |
| | | interaction, required for |
| | | neuronal maintenance |
| Trip6 | Thyroid hormone | interacts with THR in the presence |
| | receptor interactor 6 | of TH, putative co-activator for |
| | | Rel transcription factor |
| Gfer | Growth factor, erv1 (S. | sulphydryl oxidase, promotes liver |
| | cerevisiae)-like | regeneration, stimulates EGFR and |
| | (augmenter of liver | MAPK pathways |
| | regeneration) | |
| Upp | Uridine phosphorylase | Interconverts uridine and uracil, |
| | | highly expressed in transformed |
| | | cells, may produce 2-deoxy-D- |
| | | ribose, a potent angiogenic factor |
| Mdfi | MyoD family inhibitor | inhibitor of bHLH and beta- |
| | | catenin/TCF transcription factors |
| Tead2 | TEA domain 2 | transcriptional factor |
| Yap | Yes-associated 65 kD | Binds Yes, transcriptional co- |
| | | activator |
| Fhl1 | Four and a half LIM | may interact with RBP-J/Su(H) |
| Zfx | Zinc Finger X-linked | zinc finger, putative transcription |
| | | factor |
| Zfp54 | Zinc finger 54 | zinc finger, putative transcription |
| | | factor |
| | Zinc finger protein | zinc finger, putative transcription |
| | | factor |
| D17Ertd197e | D17Ertd197e | zinc finger, putative transcription |
| | | factor |
| | ESTs, high similarity | zinc finger, putative transcription |
| | to Zfp | factor |
| | ESTs, high similarity | zinc finger, putative transcription |
| | to Zfp | factor |
| | ESTs, high similarity | zinc finger, putative transcription |
| | to Zfp | factor |
| Rnf4 | RING finger 4 | steroid-mediated transcription |
| Chd1 | Chromodomain | modification of chromatin |
| | helicase DNA binding | structure, SNF2/SW12 family |
| | protein 1 | |
| Etl1 | enhancer trap locus 1 | modification of chromatin |
| | | structure, SNF2/SW12 family |
| Rmp | Rpb5-mediating pro- | Binds RNA, PolII, inhibits |
| | tein | transcription |
| Ercc5 | Excision repair 5 | Endonuclease, repair of UV- |
| | | induced damage |
| Xrcc5 | X-ray repair 5 (Ku80) | helicase, involved in V(D)J |
| | | recombination |
| Msh2 | MutS homolog 2 | mismatch repair, mutated in colon |
| | | cancer |
| Rad23b | Rad23b homolog | excision repair |
| Ccnd1 | Cyclin D1 | G1/S transition, regulates CDk2 |
| | | and 4, overexpressed in breast |
| | | cancer, implicated in other |
| | | cancers |
| Cdkn1a | Cdk inhibitor 1a P21 | inhibits G1/S transition, Cdk2 |
| | | inhibitor, required for HSC |
| | | maintenance |
| Cdkap1 | Cdk2 associated pro- | binds DNA primase, possible |
| | tein | regulator of DNA replication |
| | | (S phase) |
| Cpr2 | Cell cycle progres- | overcomes G1 arrest in |
| | sion 2 | S. cerevisiae |
| Gas2 | Growth arrest | highly expressed in growth arrested |
| | specific 2 | cells, part of actin cytoskeleton |
| CenpC | Centromere protein C | present in active centromeres |
| Wig1 | Wild-type p53 | p53 target, inhibits tumor cell |
| | induced 1 | growth |
| Tmk | Thymidylate kinase | dTTP synthesis pathway, essential |
| | | for S phase progression |
| Umps | Uridine mono- | Pyrimidine biosynthesis |
| | phosphate | |
| | synthetase | |
| Sfrs3 | Splicing factor RS | implicated in tissue-specific |
| | rich 3 | differential splicing, cell cycle |
| | | regulated |
| | ESTs highly similar | Cell cycle-regulated nuclear |
| | to exportin 1 | export protein |
| | ESTs highly similar | trifunctional protein of pyrimidine |
| | to CAD | biosynthesis, activated |
| | | (phosphorylated) by MAPK |
| | ESTs similar to | Map kinase cascade |
| | Mapkkkk3 | |
| Gas2 | Growth arrest | highly expressed in growth arrested |
| | specific 2 | cells, part of actin cytoskeleton, |
| | | target of caspase-3, stabilizes p53 |
| Wig1 | Wild-type p53 | p53 target, inhibits tumor cell |
| | induced 1 | growth |
| Pdcd2 | Programmed cell | Unknown |
| | death 2 | |
| Sfrs3 | Splicing factor RS | implicated in tissue-specific |
| | rich 3 | differential splicing, cell cycle |
| | | regulated |
| | ESTs highly similar | putative splicing factor |
| | to Sfrs6 | |
| | ESTs highly similar | putative splicing factor |
| | to pre-mRNA splicing | |
| | factor Prp6 | |
| Snrp1c | Small nuclear | U1 snRNPs, component of the |
| | ribonucleoprotein | spliceosome |
| | polypeptide C | |
| Phax | Phosphorylated | mediates U snRNA nuclear export |
| | adaptor for | |
| | RNA export | |
| NOL5 | Nucleolar protein 5 | pre-rRNA processing |
| | (SIK similar) | |
| | ESTs highly similar | pre-rRNA processing |
| | to Nop56 | |
| Rnac | RNA cyclase | Unknown |
| | ESTs highly similar | DEAD-box protein, putative RNA |
| | to Ddx1 | helicase |
| Eif4ebp1 | Eukaryotic translation | translational repressor, regulated |
| | initiation factor 4E | (phosphorylated) by several |
| | binding protein 1 | signaling pathways |
| Eif4g2 | Eukaryotic translation | translational repressor, required |
| | initiation factor 4, | for gastrulation and ESC |
| | gamma 2 | differentiation |
| | ESTs highly similar | Translation initiation factor |
| | to Eif3s1 | |
| Mrps31 | Mitochondrial | component of the ribosome, |
| | ribosomal protein S31 | mitochondria |
| Mrpl17 | Mitochondrial | component of the ribosome, |
| | ribosomal protein L17 | mitochondria |
| Mrpl34 | Mitochondrial | component of the ribosome, |
| | ribosomal protein L34 | mitochondria |
| Hspal1 | Heat shock 70 kD | Chaperone, testis-specific |
| | protein-like 1 (Hsc70t) | |
| Hspa4 | Heat shock 70 kDa | Chaperone |
| | protein 4 (Hsp110) | |
| Dnajb6 | DnaJ (Hsp40) homo- | co-chaperone |
| | log, subfamily B, | |
| | member 6 | |
| | (Mammalian rela- | |
| | tive of Dnaj) | |
| Hrsp12 | Heat responsive | possible chaperone |
| Tcp1-rs1 | T-complex protein 1 | possible chaperone |
| | related sequence 1 | |
| Ppic | Peptidylprolyl | Isomerization of peptidyl-prolyl |
| | isomerase C | bonds |
| | (cyclophilin C) | |
| Fkbp9 | FK506-binding protein | possible peptidyl-prolyl isomerase |
| | 9 (63 kD) | |
| | ESTs moderately | possible peptidyl-prolyl isomerase |
| | similar to Fkbp13 | |
| Ube2d2 | Ubiquitin-conjugating | E2, Ubiquitination of proteins |
| | enzyme E2D2 | |
| Arih1 | Ariadne homolog | likely E3, Ubiquitin ligase |
| Fbxo8 | F-box only 8 | putative SCF Ubiquitin ligase |
| | | subunit |
| | ESTs moderately | possible E2, Ubiquitination of |
| | similar to Ubc13 | proteins |
| | (bendless) | |
| Usp9x | Ubiquitin protease 9, | removes ubiquitin from proteins |
| | X chromosome | |
| Uchrp | Ubiquitin c-terminal | likely removes ubiquitin from |
| | hydrolase related | proteins |
| | polypeptide | |
| Axo | Axotrophin | contains RING-CH domain similar |
| | | to E3s, Ubiquitin ligases |
| Tpp2 | Tripeptidyl peptidase | serine expopeptidase, associated |
| | II | with the proteasome |
| Cops4 | COP9 (constitutive | Cop9 signalosome, integration of |
| | photomorphogenic) | multiple signaling pathways, |
| | subunit 4 | regulation of protein degradation |
| Cops 7a | COP9 (constitutive | Cop9 signalosome, integration of |
| | photomorphogenic), | multiple signaling pathways, |
| | subunit 7a | regulation of protein degradation |
| | ESTs highly similar | Regulatory subunit of the |
| | to proteasome 26S | proteasome |
| | subunit, non-ATPase, | |
| | 12 (p55) | |
| Nyren18 | NY-REN-18 antigen | interferon-9 induced, |
| | (NUB1) | downregulator of Nedd8, a |
| | | ubiquitin-like protein |
| Rab18 | Rab18, member RAS | small GTPase, may regulate |
| | oncogene family | vesicle transport |
| Rabggtb | RAB geranlygeranyl | regulates membrane association |
| | transferase, b subunit | of Rab proteins |
| Stxbp3 | Syntaxin binding | vesicle/membrane fusion |
| | protein 3 | |
| Sec23a | Sec23a (S. cerevisiae) | ER to Golgi transport |
| | ESTs moderately | ER to Golgi transport |
| | similar to Coatomer | |
| | delta | |
| Abcb1 | Multi-drug resistance 1 | exclusion of toxic chemicals |
| | (Mdr1) | |
| Gsta4 | Glutathione S- | response to oxidative stress |
| | transferase 4 | |
| Gslm | Glutamate-cycteine | glutathione biosynthesis |
| | ligase modifier subunit | |
| Txnrd1 | Thioredoxin reductase | delivers reducing equivalents |
| | | to Thioredoxin |
| Txn1 | Thioredoxin-like 32 | redox balance, reduces |
| | kD | dissulphide bridges in proteins |
| Laptm4a | Lysosomal-associated | import of small molecules into |
| | protein transmembrane | lysosome |
| | 4A (MTP) | |
| Rcn | Reticulocalbin | ER protein, Ca+2 binding, |
| | | overexpressed in tumor cell lines |
| Supl15h | Suppressor of Lec15 | ER synthesis of dolichol |
| | homolog | phosphate-mannose, precursor to |
| | | GPI anchors and N-glycosylation |
| Pla2g6 | Phospholipase A2, | Hydrolysis of phospholipids |
| | group VI | |
| Acadm | Acetyl-Coenzyme A | fatty acid beta-oxidation |
| | dehydrogenase, | |
| | medium chain | |
| Suclg2 | Succinate-Coenzyme | regulatory subunit, Krebs cycle |
| | A ligase, GDP- | |
| | forming, beta sub- | |
| | unit | |
| Pex7 | Peroxisome biogenesis | Peroxisomal protein import |
| | factor 7 | receptor |
| Gcat | Glycine C- | conversion of threonine to |
| | acetyltransferase | glycine |
| | (KBL) | |
| Tjp1 | Tight junction | component of tight junctions, |
| | protein 1 | interacts with cadherins in |
| | | cells lacking tight junctions |

In one embodiment, reprogramming factors can be delivered to a cell of interest using any suitable method known in the art including but not limited to transfection, transfection with liposomes, transfection with chemical compositions, and stable integration into a genome. Transfection is typically achieved through integrating viral vectors, such as retroviruses and lentiviruses. Recombinant retroviruses such as the Moloney murine leukemia virus have the ability to integrate into the host genome in a stable fashion. They contain a reverse transcriptase which allows integration into the host genome.

Lentiviruses are a subclass of Retroviruses. They are widely adapted as vectors thanks to their ability to integrate into the genome of non-dividing as well as dividing cells. These viral vectors also have been widely used in a broader context: differentiation programming of cells, including dedifferentiation, differentiation, and transdifferentiation. The viral genome in the form of RNA is reverse-transcribed when the virus enters the cell to produce DNA, which is then inserted into the genome at a random position by the viral integrase enzyme.

In another embodiment, methods essentially free of exogenous genetic elements, such as from retroviral or lentiviral vectors, can be used to deliver the gene of interest to a cell. These methods make use of extra-chromosomally replicating vectors, or vectors capable of replicating episomally. A number of DNA viruses, such as adenoviruses, Simian vacuolating virus 40 (SV40) or bovine papilloma virus (BPV), or budding yeast ARS (Autonomously Replicating Sequences)-containing plasmids replicate extra-chromosomally or episomally in mammalian cells. These episomal plasmids are intrinsically free from all these disadvantages (Bode et al., 2001) associated with integrating vectors but have never been publicly disclosed for generating induced pluripotent stem cells. A lymphotrophic herpes virus-based including or Epstein Barr Virus (EBV) as defined above may also replicate extra-chromosomally and help deliver reprogramming genes to somatic cells. Although the replication origins of these viruses or ARS element are well characterized, they have never been known for reprogramming differentiated cells to public until this disclosure.

A Reprogrammed Cell

The invention provides a reprogrammed cell that is obtained in the absence of eggs, embryos, embryonic stem cells, or somatic cell nuclear transfer (SCNT). A reprogrammed cell produced by the methods of the invention may be pluripotent or multipotent. A reprogrammed cell produced by the methods of the invention can have a variety of different properties including embryonic stem cell like properties. For example, a reprogrammed cell may be capable of proliferating for at least 10, 15, 20, 30, or more passages in an undifferentiated state. In other forms, a reprogrammed cell can proliferate for more than a year without differentiating. Reprogrammed cells can also maintain a normal karyotype while proliferating and/or differentiating. Some reprogrammed cells also can be cells capable of indefinite proliferation in vitro in an undifferentiated state. Some reprogrammed cells also can maintain a normal karyotype through prolonged culture. Some reprogrammed cells can maintain the potential to differentiate to derivatives of all three embryonic germ layers (endoderm, mesoderm, and ectoderm) even after prolonged culture. Some reprogrammed cells can form any cell type in the organism. Some reprogrammed cells can form embryoid bodies under certain conditions, such as growth on media that do not maintain undifferentiated growth. Some reprogrammed cells can form chimeras through fusion with a blastocyst, for example.

Reprogrammed cells can be defined by a variety of markers. For example, some reprogrammed cells express alkaline phosphatase. Some reprogrammed cells express SSEA-1, SSEA-3, SSEA-4, TRA-1-60, and/or TRA-1-81. Some reprogrammed cells express Oct 4, Sox2, and Nanog. It is understood that some reprogrammed cells will express these at the mRNA level and still others will also express them at the protein level, on for example, the cell surface or within the cell.

A reprogrammed cell can have any combination of any reprogrammed cell property or category or categories and properties discussed herein. For example, a reprogrammed cell can express alkaline phosphatase, not express SSEA-1, proliferate for at least 20 passages, and be capable of differentiating into any cell type. Another reprogrammed cell, for example, can express SSEA-1 on the cell surface, and be capable of forming endoderm, mesoderm, and ectoderm tissue and be cultured for over a year without differentiation.

A reprogrammed cell can be alkaline phosphatase (AP) positive, SSEA-1 positive, and SSEA-4 negative. A reprogrammed cell also can be Nanog positive, Sox2 positive, and Oct-4 positive. A reprogrammed cell also can be Tell positive, and Tbx3 positive. A reprogrammed cell can also be Cripto positive, Stellar positive and Daz1 positive. A reprogrammed cell can express cell surface antigens that bind with antibodies having the binding specificity of monoclonal antibodies TRA-1-60 (ATCC HB-4783) and TRA-1-81 (ATCC HB-4784). Further, as disclosed herein, a reprogrammed cell can be maintained without a feeder layer for at least 10, 11, 12, 13, 14, 15, 16, 17, 18, 19, 20 passages or for over a year.

A reprogrammed cell may have the potential to differentiate into a wide variety of cell types of different lineages including fibroblasts, osteoblasts, chondrocytes, adipocytes, skeletal muscle, endothelium, stroma, smooth muscle, cardiac muscle, neural cells, hemiopoetic cells, pancreatic islet, or virtually any cell of the body. A reprogrammed cell may have the potential to differentiate into all cell lineages. A reprogrammed cell may have the potential to differentiate into any number of lineages including 1, 2, 3, 4, 5, 6-10, 11-20, 21-30, and greater than 30 lineages.

Embodiments of the invention also include methods for treating a variety of diseases using a reprogrammed cell produced according to the novel methods disclosed elsewhere herein. The skilled artisan would appreciate, based upon the disclosure provided herein, the value and potential of regenerative medicine in treating a wide plethora of diseases including, but not limited to, heart disease, diabetes, skin diseases and skin grafts, spinal cord injuries, Parkinson's disease, multiple sclerosis, Alzheimer's disease, and the like. The present invention encompasses methods for administering reprogrammed cells to an animal, including humans, in order to treat diseases where the introduction of new, undamaged cells will provide some form of therapeutic relief.

Embodiments of the invention relate to the use of reprogrammed cells and reprogrammed cell lines produced using the methods disclosed herein for drug screening and toxicity assays. In another embodiment, the invention relates to the use of a cell culture of reprogrammed cells for drug screening and toxicity assays.

In another embodiment, the invention relates to reagents and methods for in vitro screening of pharmaceutical and non-pharmaceutical chemicals using induced pluripotent or multipotent stem cells or iPS-derived lineage-specific cells, such as neural stem cells, muscle cells, and pancreatic cells. In yet another embodiment, the invention relates to methods for in vitro screening of toxicity and teratogenicity of chemical compounds using induced pluripotent or multipotent stem cells or iPS-derived lineage-specific cells.

In another embodiment, the invention provides human-specific in vitro methods for reliably determining toxicity of pharmaceuticals and other chemical compounds using induced pluripotent or multipotent stem cells or iPS-derived lineage-specific cells, thus overcoming the limitations associated with interspecies animal models. The methods can be used to test compounds that can produce positive, negative or a combination of positive and negative affects on the cell. Compounds can be tested on lineage specific cells, including but not limited to neuronal cells, muscle cell, cardiac cells, and pancreatic cells.

In another embodiment, the invention provides methods for using undifferentiated induced pluripotent stem cells or iPS-derived lineage-specific cells for in vitro evaluation. In one embodiment, undifferentiated induced pluripotent or multipotent stem cells or iPS-derived lineage-specific cells, are exposed to test compounds at any concentration including but not limited to concentrations reflective of in vivo levels.

Further embodiments of this aspect of the invention provide for determination of the capacity of the test compound to induce differentiation of induced pluripotent or multipotent stem cells into particular cell types.

In another embodiment, the invention relates to methods for using pluripotent, non-lineage restricted cells to screen compounds. The benefit of utilizing pluripotent stem cells is they permit analysis of global toxic response(s) and are isolated from the physiological target of developmental toxicity. In addition, because these cells have not differentiated into a specific lineage, the potential for false negatives is reduced.

In another embodiment, the invention relates to methods for identifying predictive biomarkers of toxic responses to chemical compounds, particularly pharmaceutical and non-pharmaceutical chemicals, and particularly to known teratogens. In embodiments of this aspect, a dynamic set representative of a plurality of cellular metabolites, preferably secreted or excreted by hES cells is determined and correlated with health and disease or toxic insult state. Cellular metabolites according to this aspect of the invention generally range from about 10 to about 1500 Daltons, more particularly from about 100 to about 1000 Daltons, and include but are not limited to compounds such as sugars, organic acids, amino acids, fatty acids and signaling low-molecular weight compounds. Said biomarker profiles are diagnostic for toxicity of chemical compounds, particularly pharmaceutical and non-pharmaceutical chemicals, that participate in and reveal functional mechanisms of cellular response to pathological or toxic chemical insult, thus serving as biomarkers of disease or toxic response that can be detected in biological fluids. In particularly preferred embodiments of this aspect of the invention, these biomarkers are useful for identifying active (or activated) metabolic pathways following molecular changes predicted, inter alia, by other methods (such as transcriptomics and proteomics).

Embodiments of the invention also relate to a method for monitoring or identifying the early stages or the initiation of reprogramming. In one embodiment, cells overexpressing a transcription factor in conjunction with a reporter construct can be used to monitor the early stages of reprogramming.

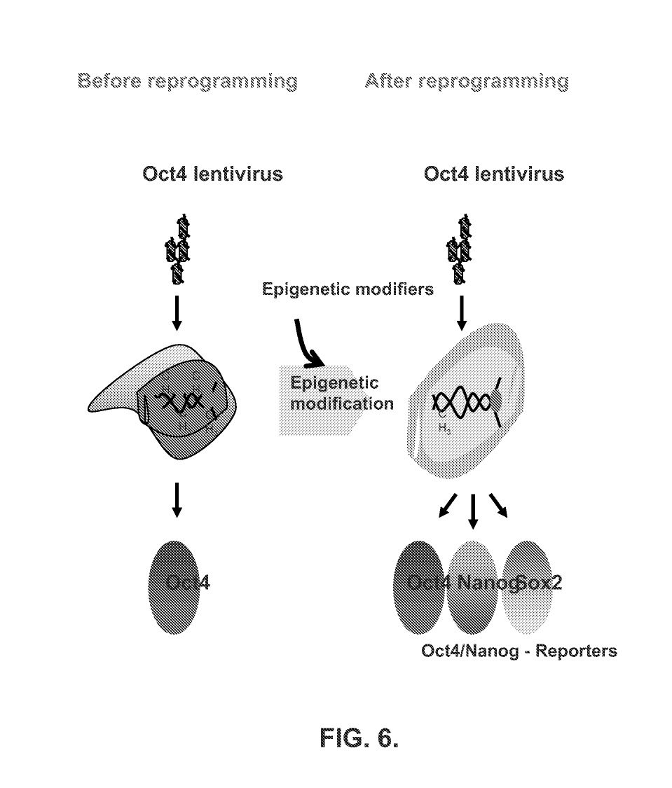

In another embodiment, the invention relates to a method comprising delivering a transcription factor to a cell, exposing said cell to a reporter construct, exposing said cell to an agent that inhibits the activity, expression or activity and expression of a gene, which codes for a protein, or a protein involved in transcriptional repression, and monitoring the output of the reporter construct (see FIG. 6). The output from the report construct is used to determine the degree or magnitude of reprogramming. In another embodiment, the reporter construct is designed to monitor the activity of a transcription factor, including but not limited to Oct-2, Sox-2 and Nanog. Any type of reporter can be used including but not limited to GFP, luciferase, alkaline phosphatase, and CAT.

The skilled artisan will readily understand that reprogrammed cells can be administered to an animal as a re-differentiated cell, for example, a neuron, and will be useful in replacing diseased or damaged neurons in the animal. Additionally, a reprogrammed cell can be administered to the animal and upon receiving signals and cues from the surrounding milieu, can re-differentiate into a desired cell type dictated by the neighboring cellular milieu. Alternatively, the cell can be re-differentiated in vitro and the differentiated cell can be administered to a mammal in need there of.

The reprogrammed cells can be prepared for grafting to ensure long term survival in the in vivo environment. For example, cells can be propagated in a suitable culture medium, such as progenitor medium, for growth and maintenance of the cells and allowed to grow to confluence. The cells are loosened from the culture substrate using, for example, a buffered solution such as phosphate buffered saline (PBS) containing 0.05% trypsin supplemented with 1 mg/ml of glucose; 0.1 mg/ml of MgCl₂, 0.1 mg/ml CaCl₂ (complete PBS) plus 5% serum to inactivate trypsin. The cells can be washed with PBS using centrifugation and are then resuspended in the complete PBS without trypsin and at a selected density for injection.

Formulations of a pharmaceutical composition suitable for peritoneal administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for peritoneal administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The invention also encompasses grafting reprogrammed cells in combination with other therapeutic procedures to treat disease or trauma in the body, including the CNS, PNS, skin, liver, kidney, heart, pancreas, and the like. Thus, reprogrammed cells of the invention may be co-grafted with other cells, both genetically modified and non-genetically modified cells which exert beneficial effects on the patient, such as chromaffin cells from the adrenal gland, fetal brain tissue cells and placental cells. Therefore the methods disclosed herein can be combined with other therapeutic procedures as would be understood by one skilled in the art once armed with the teachings provided herein.

The reprogrammed cells of this invention can be transplanted “naked” into patients using techniques known in the art such as those described in U.S. Pat. Nos. 5,082,670 and 5,618,531, each incorporated herein by reference, or into any other suitable site in the body.

The reprogrammed cells can be transplanted as a mixture/solution comprising of single cells or a solution comprising a suspension of a cell aggregate. Such aggregate can be approximately 10-500 micrometers in diameter, and, more preferably, about 40-50 micrometers in diameter. A reprogrammed cell aggregate can comprise about 5-100, more preferably, about 5-20, cells per sphere. The density of transplanted cells can range from about 10,000 to 1,000,000 cells per microliter, more preferably, from about 25,000 to 500,000 cells per microliter.

Transplantation of the reprogrammed cell of the invention can be accomplished using techniques well known in the art as well those developed in the future. The invention comprises a method for transplanting, grafting, infusing, or otherwise introducing reprogrammed cells into an animal, preferably, a human.

The reprogrammed cells also may be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

In one embodiment, the methods of this invention result in the derivation of endodermal cells from a cell differentiated from an induced pluripotent stem cell (an iPS cell).

In one embodiment, the methods of this invention result in the derivation of mesodermal cells from a cell differentiated from an iPS cell.

In one embodiment, the methods of this invention result in the derivation of ectodermal cells from a cell differentiated from an iPS cell.

In one embodiment, the methods of this invention result in the derivation of neuroglial precursor cells from a cell differentiated from an iPS cell.

In one embodiment, the methods of this invention result in the derivation of hepatic cells or hepatic precursor cells from a cell differentiated from an iPS cell.

In one embodiment, the methods of this invention result in the derivation of chondrocyte or chondrocyte precursor cells from a cell differentiated from an iPS cell.

In one embodiment, the methods of this invention result in the derivation of myocardial or myocardial precursor cells from a cell differentiated from an iPS cell.

In one embodiment, the methods of this invention result in the derivation of gingival fibroblast or gingival fibroblast precursor cells from a cell differentiated from an iPS cell.

In one embodiment, the methods of this invention result in the derivation of pancreatic beta cells or pancreatic beta precursor cells from a cell differentiated from an iPS cell.

In one embodiment, the methods of this invention result in the derivation of retinal precursor cells with from a cell differentiated from an iPS cell.

In one embodiment, the methods of this invention result in the derivation of hemangioblasts from a cell differentiated from an iPS cell.

In one embodiment, the methods of this invention result in the derivation of dermal fibroblasts with prenatal patterns of gene expression from a cell differentiated from an iPS cell.

Reprogrammed cells of the invention also can be used to express a foreign protein or molecule for a therapeutic purpose or for a method of tracking their integration and differentiation in a patient's tissue. Thus, the invention encompasses expression vectors and methods for the introduction of exogenous DNA into reprogrammed cells with concomitant expression of the exogenous DNA in the reprogrammed cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Embodiments of the invention also relate to a method for identifying regulators of the epigenome comprising contacting a cell with a small molecule library, measuring a change to the genome; and identifying the regulator of the genome. The method further comprises identifying the small molecule modulator. In still another embodiment, measuring a change to the genome includes but is not limited to acetylation, deacetylation, methylation, demethylation, phosphorylation, ubiquitination, sumoylation, ADP-ribosylation, and deimination.

Embodiments of the invention also relate to a composition comprising a cell that has been produced by the methods of the invention. In another embodiment, the invention relates to a composition comprising cell that has been reprogrammed by delivering a transcription factor and exposing said cell to an agent that inhibits the activity, expression, or activity and expression of a gene, which codes for a protein, or a protein involved in transcriptional respression, and inducing somatic cell reprogramming factors through endogenous auto- and reciprocal transcriptional regulation. In yet another embodiment, the invention relates to a composition comprising a cell that has been reprogrammed by delivering a single transcription factor.

Embodiments of the invention also relate to a reprogrammed cell that has been produced by contacting a cell with a single transcription factor and exposing said cell to an agent that inhibits the activity, expression or activity and expression of a gene, which codes for a protein, or a protein involved in transcriptional repression.

Kits

Embodiments of the invention also relate to kits for preparing the methods and compositions of the invention. The kit can be used for, among other things, producing a reprogrammed cell and generating ES-like and stem cell-like cells, overexpressing a transcription factor, exposing said cell to an agent that inhibits the activity, expression or activity and expression of a gene, which codes for a protein, or a protein involved in transcriptional repression, inducing the expression of a gene that contributes to a cell being pluripotent or multipotent. The kit may comprise at least one agent for overexpression of a transcription factor. The kit may comprise multiple agents for overexpression of transcription factors. The agents for overexpression of a transcription factor can be provided in a single container or in multiple containers.

The kit may also comprise reagents necessary to determine if the cell has been reprogrammed including but not limited to reagents to test for the induction of a gene that contributes to a cell being pluripotent or multipotent, reagents to test for inhibition of a DNMT, regents to test for demethylating of CpG dinucleotides, and reagents to test for remodeling of the chromatin structure.

The kit may also comprise regents that can be used to differentiate the reprogrammed cell into a particular lineage or multiple lineages including but not limited to a neuron, an osteoblast, a muscle cell, an epithelial cell, and hepatic cell.

The kit may also contain an instructional material, which describes the use of the components provide in the kit. As used herein, an “instructional material” includes a publication, a recording, a diagram, or any other medium of expression that can be used to communicate the usefulness of the methods of the invention in the kit for, among other things, effecting the reprogramming of a differentiated cell. Optionally, or alternately, the instructional material may describe one or more methods of re- and/or trans-differentiating the cells of the invention. The instructional material of the kit of the invention may, for example, be affixed to a container that contains a small molecule inhibitor. Alternatively, the instructional material may be shipped separately from the container with the intention that the instructional material and a small molecule inhibitor, or component thereof, be used cooperatively by the recipient.

The invention is now described with reference to the following Examples. These Examples are provided for the purpose of illustration only and the invention should in no way be construed as being limited to these Examples, but rather should be construed to encompass any and all variations that become evident as a result of the teaching provided herein. All references including but not limited to U.S. patents, allowed U.S. patent applications, or published U.S. patent applications are incorporated within this specification by reference in their entirety.

### EXAMPLES

The following examples are illustrative only and are not intended to limit the scope of the invention as defined by the claims.

### Example 1

RNA interference through siRNA or recently developed shRNA provides specific gene knockdown in vitro and in vivo. However, the efficiency of gene silencing is dependent on the delivery system and host cell tropism. The use of a retroviral or lentiviral vector dramatically enhances efficiency of transfection into a wide range of mammalian cell types in culture compared to traditional chemical delivery systems. Furthermore, additional selection and visualization is possible by adding antibiotic resistance genes and GFP, respectively, to the viral vector. In this example, we explored the transfection efficiency for different cell types.

Methods

Cell Culture.

Human dermal fibroblasts or human dermal fibroblasts harboring an Oct4-GFP reporter, were maintained at 37° C. in 95% humidity and 5% CO₂ in Dulbecco's modified eagle medium (DMEM, Cell Application) containing 10% fetal bovine serum, 0.5% penicillin and streptomycin and additional zeocin (25 μg/ml) or puromycin (2 μg/ml) as needed for the selection. Cells were grown, trypsinized and counted, then diluted in the above standard growth medium to achieve appropriate plating density prior to introduction of shRNA or transcription factor lentivirus.

Lentivirus Infection.

To determine transfection efficiency of different origins of fibroblast cultures, ten transfection units of GAPDH-targeting shRNA lentiviral particles were infected into primary cultures from human subcutaneous adipose tissue (Preadipocytes), lung (HLF), skeletal muscle (SkM) and skin (HDF). High tittered SMARTvector™ shRNA lentivirus (≧10⁸ Transfection Unit/ml) for epigenetic modification was obtained from Dharmacon (Thermo Fisher Scientific, www.dharmacon.com). Highly functional long-term gene silencing technology from Dharmacon designed for green fluorescene protein (turboGFP™) visualization and puromycin selection was utilized. Ultra-high tittered lentivirus for transcription factor overexpression was obtained from SBI (System Biosciences, www.systembio.com). The day before shRNA lentiviral infection, human dermal fibroblast cells were seeded at a density of 2×10⁵ cells/ml. The next day, the medium was replaced with pre-warmed medium containing 3 μg/ml polybrene (Sigma-Aldrich) and SMARTvector™ shRNA lentivirus (5 MOI). Eighteen hours after infection, the medium was replaced with fresh medium, and lentiviral infection was repeated for transcription factor overexpression. After 2 days lentiviral infection, cells were assessed for TurboGFP (shRNA) and RFP (iPS factor) expression by fluorescence microscopy. Cells were harvested to confirm target gene knockdown and overexpression by measuring gene expression levels using quantitative real time RT-PCR and further characterization.

Quantitative RT-PCR.

Expression levels for shRNA-targeted genes and pluripotency transcription factor were quantified by real-time RT-PCR. Briefly, total RNA was prepared from cultures using Trizol Reagent (Life Technology) and the RNeasy Mini RNA isolation kit (Qiagen) with DNase I digestion according to manufacturer's protocol. Total RNA (1 μg) from each sample was subjected to oligo(dT)-primed reverse transcription (Invitrogen) to cDNA. Real-time PCR reactions were performed with PCR master mix on a 7300 real-time PCR system (Applied Biosystems). For each sample, 1 μl of diluted cDNA (1:10) was added as template in the PCR reactions. Expression levels were compared to those in untreated and non-targeting shRNA control cells relative to cyclophillin.

Results

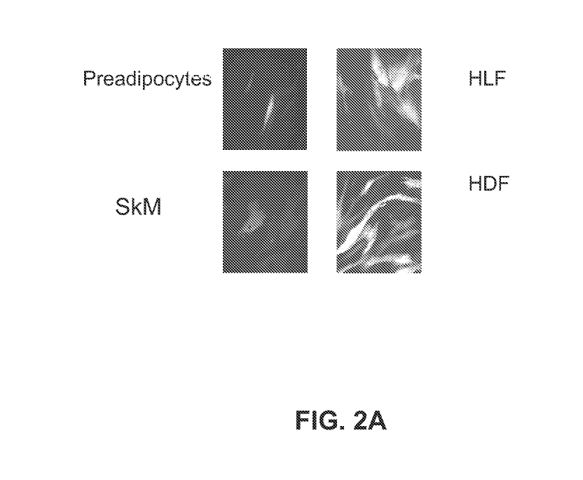

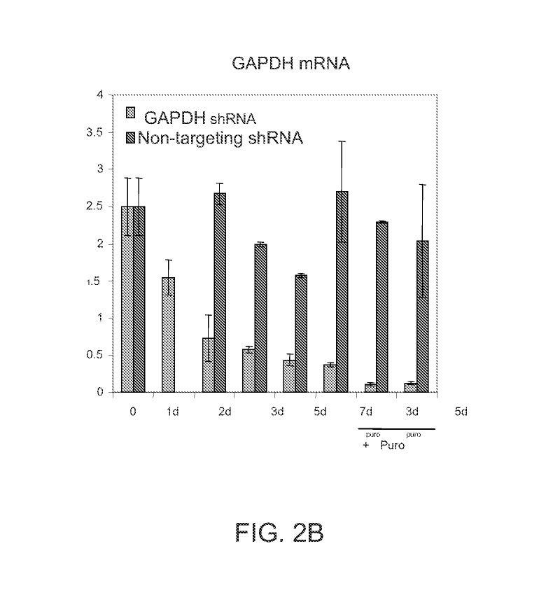

FIG. 2A illustrates that more than 80% of HDF cells were positive for turboGFP™ expression as a transfection marker. In contrast, preadipocyte and SkM cells resulted in less than 5% transfection yield. Furthermore, expression of GAPDH mRNA from HDF (FIG. 2B) suggested that greater than 80% of GAPDH expression was knocked down after 5 days of GAPDH-targeting shRNA lentiviral infection. Further knockdown of GAPDH expression with puromycin selection (3 μg/ml) was observed (FIG. 2B), resulting in more than 95% of cells expressing turboGFP™ by fluorescence microscopy (data not shown). These results indicate that the shRNA lentiviral system represents an efficient method for knocking down the expression of target genes that repress transcription of key pluripotency genes.

### Example 2

Epigenetic components including DNMTs and histone deacetylases (HDACs) play an important role in regulating transcription of development-related genes as well as reprogramming of somatic cells. As HDFs exhibited efficient lentiviral transfection efficiency (see FIG. 2A), we tested the effects of shRNA-induced knockdown of DNMT1 and HDAC on pluripotency gene expression in this cell type.

Methods

Cell Culture.

Human dermal fibroblasts or, human dermal fibroblasts harboring an Oct4-GFP reporter, were maintained at 37° C. in 95% humidity and 5% CO₂ in Dulbecco's modified eagle medium (DMEM, Cell Application) containing 10% fetal bovine serum, 0.5% penicillin and streptomycin and additional zeocin (25 μg/ml) or puromycin (2 μg/ml) as needed for the selection. Cells were grown, trypsinized and counted, then diluted in the above standard growth medium to achieve appropriate plating density prior to introduction of shRNA or transcription factor lentivirus.

Lentivirus Infection.

High tittered SMARTvector™ shRNA lentivirus (≧10⁸ Transfection Unit/ml) for epigenetic modification was obtained from Dharmacon (Thermo Fisher Scientific, www.dharmacon.com). Highly functional long-term gene silencing technology from Dharmacon designed for green fluorescence protein (turboGFP™) visualization and puromycin selection was utilized. Ultra-high tittered lentivirus for transcription factor overexpression was obtained from SBI (System Biosciences, www.systembio.com). The day before shRNA lentiviral infection, human dermal fibroblast cells were seeded at a density of 2×10⁵ cells/ml. The next day, the medium was replaced with pre-warmed medium containing 3 μg/ml polybrene (Sigma-Aldrich) and SMARTvector™ shRNA lentivirus (5 MOI). Eighteen hours after infection, the medium was replaced with fresh medium, and lentiviral infection was repeated for transcription factor overexpression. After 2 days lentiviral infection, cells were assessed for TurboGFP (shRNA) and RFP (iPS factor) expression by fluorescence microscopy. Cells were harvested to confirm target gene knockdown and overexpression by measuring gene expression levels using quantitative real time RT-PCR and further characterization.

Quantitative RT-PCR.

Expression levels for shRNA-targeted genes and pluripotency transcription factor were quantified by real-time RT-PCR. Briefly, total RNA was prepared from cultures using Trizol Reagent (Life Technology) and the RNeasy Mini RNA isolation kit (Qiagen) with DNase I digestion according to manufacturer's protocol. Total RNA (1 μg) from each sample was subjected to oligo(dT)-primed reverse transcription (Invitrogen) to cDNA. Real-time PCR reactions were performed with PCR master mix on a 7300 real-time PCR system (Applied Biosystems). For each sample, 1 μl of diluted cDNA (1:10) was added as template in the PCR reactions. Expression levels were compared to those in untreated and non-targeting shRNA control cells relative to cyclophillin.

Results

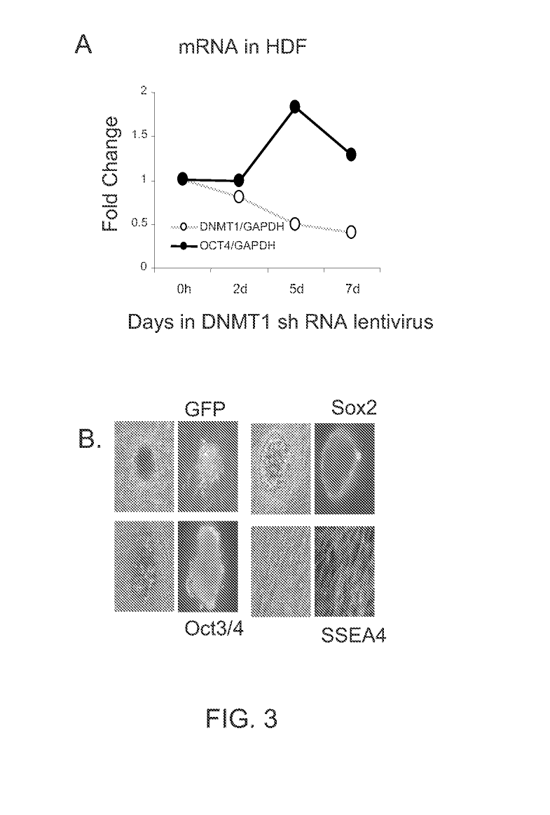

As shown in FIG. 3A, the expression of DNMT1 mRNA (open circle) was diminished by as much as 50 to 60% compared to the control after 5 days of DNMT1-targeted shRNA lentiviral infection. During this process, the expression of Oct4 (closed circle) was increased (FIG. 3A). Human ES culture medium showed no further induction of Oct4 expression (data not shown). Additional subculture with puromycin selection resulted in formation of multiple colonies exhibiting positive staining for Oct4, Sox2 and SSEA4 proteins (FIG. 3B). Similar to the colonies reported after removal of either Klf4 or cMyc, these colonies failed to proliferate and continuously grow under hES culture condition. It is possible that inhibition of a single DNMT is not sufficient to completely activate the necessary reprogramming processes. A more efficient method of reprogramming may require targeted multiple pathways or multiple components of the same pathway.

### Example 3

The effects of HDAC7 and HDAC 11 shRNA lentiviral infection on expression of pluripotency genes and other HDACs were tested. The effects of a histone deacetylase inhibitor (VPA) were also examined.

Methods

Cell Culture.

Human dermal fibroblasts or, human dermal fibroblasts harboring an Oct4-GFP reporter, were maintained at 37° C. in 95% humidity and 5% CO₂ in Dulbecco's modified eagle medium (DMEM, Cell Application) containing 10% fetal bovine serum, 0.5% penicillin and streptomycin and additional zeocin (25 ug/ml) or puromycin (2 ug/ml) as needed for the selection. Cells were grown, trypsinized and counted, then diluted in the above standard growth medium to achieve appropriate plating density prior to introduction of shRNA or transcription factor lentivirus.

Lentivirus Infection.

High tittered SMARTvector™ shRNA lentivirus (≧10⁸ Transfection Unit/ml) for epigenetic modification was obtained from Dharmacon (Thermo Fisher Scientific, www.dharmacon.com). Highly functional long-term gene silencing technology from Dharmacon designed for green fluorescene protein (turboGFP™) visualization and puromycin selection was utilized. Ultra-high tittered lentivirus for transcription factor overexpression was obtained from SBI (System Biosciences, www.systembio.com). The day before shRNA lentiviral infection, human dermal fibroblast cells were seeded at a density of 2×10⁵ cells/ml. The next day, the medium was replaced with pre-warmed medium containing 3 μg/ml polybrene (Sigma-Aldrich) and SMARTvector shRNA lentivirus (5 MOI). Eighteen hours after infection, the medium was replaced with fresh medium, and lentiviral infection was repeated for transcription factor overexpression. After 2 days lentiviral infection, cells were assessed for TurboGFP (shRNA) and RFP (iPS factor) expression by fluorescence microscopy. Cells were harvested to confirm target gene knockdown and overexpression by measuring gene expression levels using quantitative real time RT-PCR and further characterization.

Quantitative RT-PCR.

Expression levels for shRNA-targeted genes and pluripotency transcription factor were quantified by real-time RT-PCR. Briefly, total RNA was prepared from cultures using Trizol Reagent (Life Technology) and the RNeasy Mini RNA isolation kit (Qiagen) with DNase I digestion according to manufacturer's protocol. Total RNA (1 μg) from each sample was subjected to oligo(dT)-primed reverse transcription (Invitrogen) to cDNA. Real-time PCR reactions were performed with PCR master mix on a 7300 real-time PCR system (Applied Biosystems). For each sample, 1 μl of diluted cDNA (1:10) was added as template in the PCR reactions. Expression levels were compared to those in untreated and non-targeting shRNA control cells relative to cyclophillin.

Results

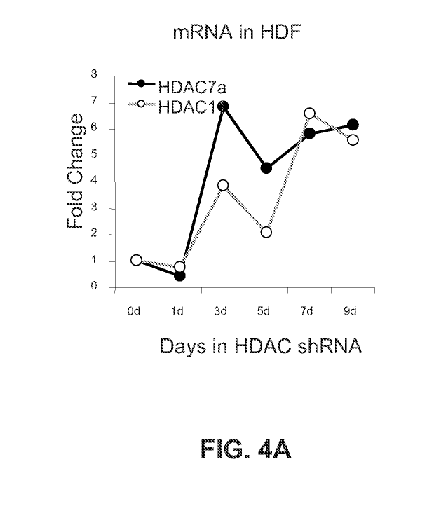

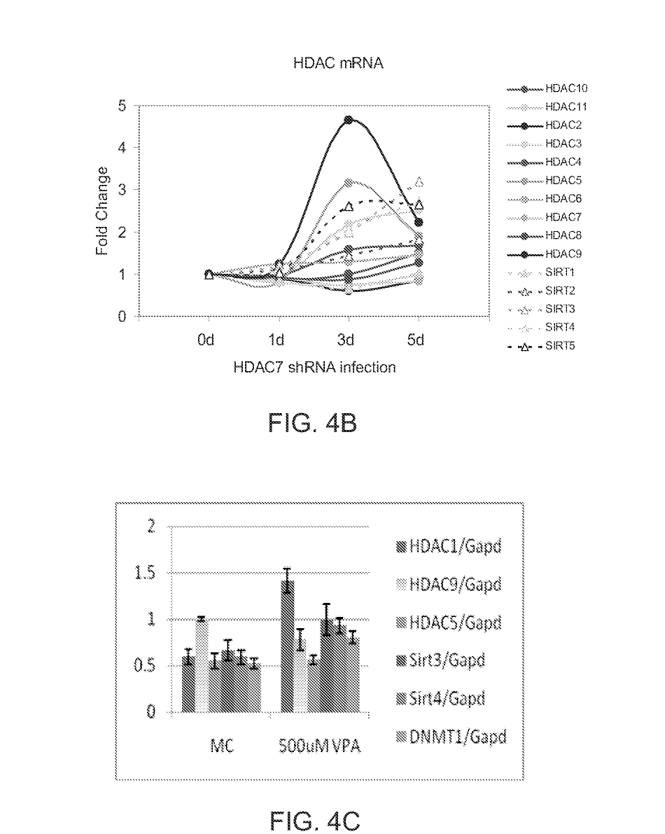

HDF cultures were infected with HDAC7 and IIDAC11-targeted shRNA lentivirus, Gene expression of Oct4 was increased slightly by HDAC7 and HDAC11 gene knockdown (<2-fold; data not shown), Nanog gene expression was significantly up regulated within 3 days after infection and this effect was maintained consistently thereafter (FIG. 4A). Immunocytochemistry also demonstrated induction of Oct4 protein in the nucleus of HDF cells with DNMT1-and/or HDAC-targeted shRNA infection (FIG. 4D).

Hyperacetylated chromatin is transcriptionally active and hypoacetylated chromatin is transcriptionally repressed. HDACs control the level of acetylated chromatin, and thus, participate in a series of pathways during cell growth and development. Interestingly, ompensatory mechanisms leading to an increase in expression of other HDACs was observed during HDAC7 gene knockdown. As shown in FIG. 4B, expression of HDAC9, HDAC5, HDAC11, SIRT4 and SIRT5 increased dramatically by 50% with HDAC7 shRNA lentiviral infection. In contrast, no compensatory induction with HDAC11 gene knockdown has been observed (data not shown). Compensatory expression for these same HDACs was also induced by DNMT1 gene knockdown (data not shown), as well as, treatment of cells with the small molecule HDAC inhibitor Valproic acid (VPA) (FIG. 4C).

These results indicate the presence of a compensatory (or redundant) pathway that likely limits efficient reprogramming. Reprogramming and induction of pluripotency markers may be effectively achieved by knocking down gene expression of multiple epi-genes, (such as DNMT combined with one or more HDACs) in human dermal fibroblasts. The observed compensation (or redundancy) is a key rate limiting step to improving reprogramming efficiency and that the identification of specific targets will focus drug discovery and design efforts aimed at replacing the current requirement of viral-based transduction methods.

### Example 4

### Methods

Cell Culture.

Human dermal fibroblasts or, human dermal fibroblasts harboring an Oct4-GFP reporter, were maintained at 37° C. in 95% humidity and 5% CO₂ in Dulbecco's modified eagle medium (DMEM, Cell Application) containing 10% fetal bovine serum, 0.5% penicillin and streptomycin and additional zeocin (25 ug/ml) or puromycin (2 ug/ml) as needed for the selection. Cells were grown, trypsinized and counted, then diluted in the above standard growth medium to achieve appropriate plating density prior to introduction of shRNA or transcription factor lentivirus.

Lentivirus Infection.

High tittered SMARTvector™ shRNA lentivirus (≧10⁸ Transfection Unit/imp for epigenetic modification was obtained from Dharmacon (Thermo Fisher Scientific, www.dharmacon.com). Highly functional long-term gene silencing technology from Dharmacon designed for green fluorescene protein (turboGFP™) visualization and puromycin selection was utilized. Ultra-high tittered lentivirus for transcription factor overexpression was obtained from SBI (System Biosciences, www.systembio.com). The day before shRNA lentiviral infection, human dermal fibroblast cells were seeded at a density of 2×10⁵ cells/ml. The next day, the medium was replaced with pre-warmed medium containing 3 μg/ml polybrene (Sigma-Aldrich) and SMARTvector™ shRNA lentivirus (5 M01). Eighteen hours after infection, the medium was replaced with fresh medium, and lentiviral infection was repeated for transcription factor overexpression. After 2 days lentiviral infection, cells were assessed for TurboGFP (shRNA) and RFP (iPS factor) expression by fluorescence microscopy. Cells were harvested to confirm target gene knockdown and overexpression by measuring gene expression levels using quantitative real time RT-PCR and further characterization.

Quantitative RT-PCR.

Expression levels for shRNA-targeted genes and pluripotency transcription factor were quantified by real-time RT-PCR. Briefly, total RNA was prepared from cultures using Trizol Reagent (Life Technology) and the RNeasy Mini RNA isolation kit (Qiagen) with DNase I digestion according to manufacturer's protocol. Total RNA (1 μg) from each sample was subjected to oligo(dT)-primed reverse transcription (Invitrogen) to cDNA. Real-time PCR reactions were performed with PCR master mix on a 7300 real-time PCR system (Applied Biosystems). For each sample, 1 id of diluted cDNA (1:10) was added as template in the PCR reactions. Expression levels were compared to those in untreated and non-targeting shRNA control cells relative to cyclophillin.

Results

Core pluripotency transcription factors (Nanog, Oct4 and Sox2) co-occupy their own promoters as well as the promoters of other factors. These interactions are further enhanced by protein-protein interactions and, as a result, activate transcription reciprocally. Such a unique transcription factor network is crucial to maintaining self-renewal and pluripotency of embryonic stem cells. Chromatin modifications, such as DNA methylation and histone modifications, distinguish the promoter regions of pluripotency transcription factors between ES cells and human dermal fibroblasts. Re-expression of silenced pluripotency genes requires extensive chromatin remodeling. Chromatin remodeling may be considered a functional definition of reprogramming. As a result, re-expression of silenced pluripotency genes requires time.

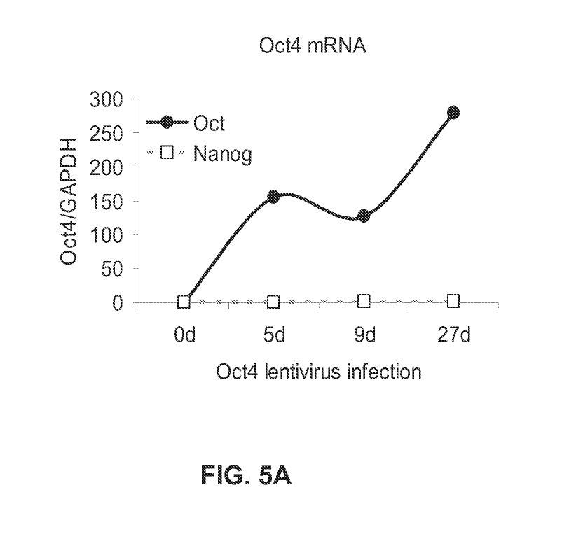

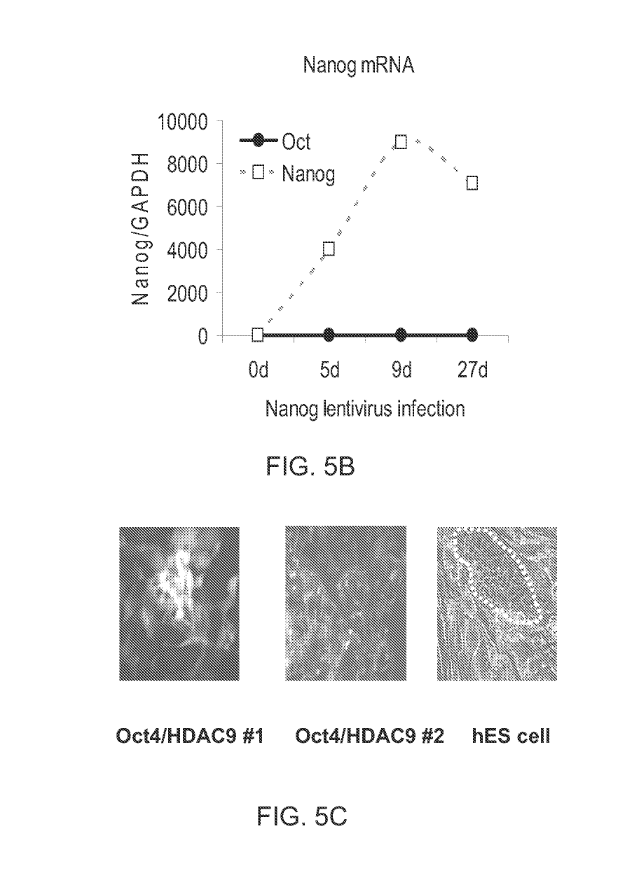

As shown in FIG. 5A and FIG. 5B, Oct4-mediated transcriptional activation of Nanog was not observed. Similarly Nanog-mediated transcriptional activation of Oct4 mRNA induction was not observed.

However, expression of lentivirus Oct-4 lead to a substantial increase in the amount of Oct-4 detected. Likewise, expression of lentivirus Nanog led to a substantial increase in the amount of Nanog detected.

Oct4 expression combined with HDAC9 shRNA lentiviral infection into HDF induced formation of multiple colonies that continuously proliferated on the MEF feeder layer, similar to hES cells (FIG. 5C). These results indicate that chromatin modification by knocking down expression of a gene that codes for a protein involved in transcriptional repression including but not limitied to an HDAC or DNMT, in conjunction with expression a single pluripotent transcription factor induces reprogramming of human dermal fibroblasts to a pluripotent stem cell-like state. Moreover, the identification of specific epi-targets that enhance reprogramming provides a basis for drug discovery and/or rational design in order to develop a purely chemical approach to induce reprogramming of a somatic nucleus to a less differentiated state.

### Example 5

Direct reprogramming of somatic cells to induced pluripotent stem (iPS) cells has been demonstrated by forced expression of one of two combinations of four transcription factors: (1) Oct-4 (O), Sox2 (5), Klf4 (K), and c-Myc (M) or (2) Oct-4 (O), Sox2 (S), Nanog and Lin28. (Takahashi and Yamanaka, 2006; Yu, Frame et al., 2007). Reprogramming efficiencies using forced expression of the above-factors is often low, and thus, methods that improve reprogramming efficiency are needed.

Methods

Cell Culture.

Human adipose tissue derived adult stem cells were maintained at 37° C. in 95% humidity and 5% CO₂ in Dulbecco's modified eagle medium (DMEM, Cell Application) containing 10% fetal bovine serum, 0.5% penicillin and streptomycin. Cells were grown, trypsinized and counted, then diluted in the above standard growth medium to achieve appropriate plating density (1×10⁵ cells/well in 6 well plate, Corning) prior to HDAC inhibitor treatment.

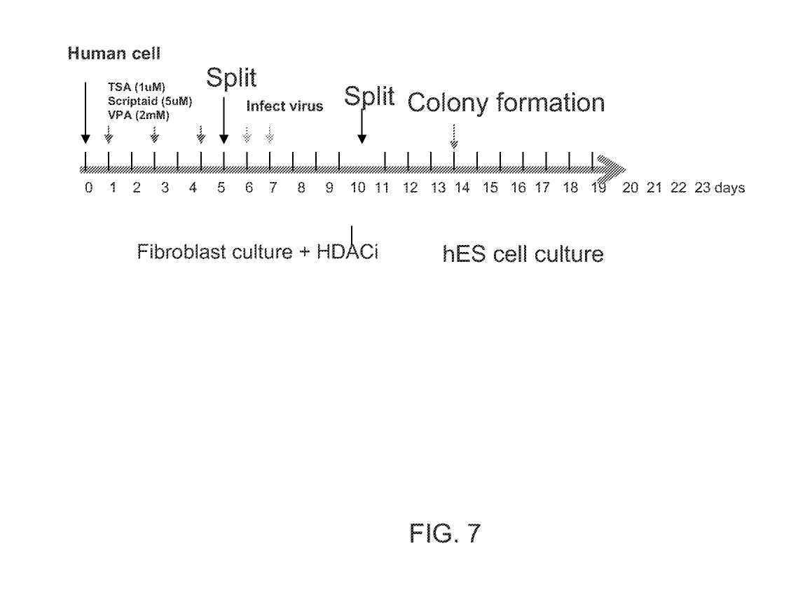

Pre-treatment. Human adipose tissue derived adult stem cells were cultured in the presence of Scriptaid, Triehostatin A, or Valproic acid for 5 days as shown in FIG. 7. The following concentrations were used: Scriptaid at 5 μM; TSA at 1 μM, and valproic acid at 2 mM. The cell culture medium was changed every other day with new treatment.

Lentivirus infection. Lentivirus that over-expresses mouse Oct-4, Sox2, c-Myc, and Klf4 was purchased from Millipore, (Billerica, Mass.). One day before lentivirus infection, human adipose tissue-derived cells with or without HDAC inhibitor pretreatment were trypsinized and plated at a density of 1×10⁵ cells/well in a 6 well plate (Corning) using standard growth medium described above. The following day, the medium was replaced with 1 ml of fresh medium containing 0.6 ug/ml of polybrene (Sigma-Aldrich, St. Louis, Mo.), and thawed lentivirus (MOI 75) was added directly to the wells following the manufacturer's instruction. Cells were maintained at 37° C. in 95% humidity and 5% CO₂ incubator overnight. On the next day, lentiviral infection was repeated after cells were washed 3 times with 1×PBS. Lentivirus infected cells were maintained in the standard growth medium for 4˜5 days and trypsinized, and plated on irradiated MEF feeder layers (ATCC, Manassas, Va.) with mTeSR medium (Stem Cell Technology).

Immunohistochemistry.

Cells were fixed with fixative (Ethanol:acetic acid:H₂O=7:2:1) for 10 minutes. After rinse 3 times with 10% FBS in 1×PBS for 5 min each and cells were incubated I hrs with primary antibodies for Oct4 (1:200 dilution, Abeam), or Sox2 (1:200 dilution, Abeam) in 10% FBS containing 0.2% saponin (Sigma) and following alexaFluor-conjugated secondary antibody (1:200 dilution, Invitrogen).

Results

A representative pre-treatment schedule is shown in FIG. 7. One of ordinary in the art will understand that the culture times and concentrations of agents may vary.

By way of representative example only, and not to limit the methods disclosed herein in any manner, cells can be cultured in the presence of an agent that reduces the activity, expression, or activity and expression of a gene that codes for a protein involved in transcriptional repression or reduces the activity of a protein involved in transcriptional repression. As shown in FIG. 7, a histone deacteylase inhibitor may be used. The cell culture medium can be changed every other day.

Following several day of pre-treatment, such as on day 7 and 8, the cells can be transfected with a virus comprising one or more than one pluripotency gene. The cells can be transfected once or multiple times.

Several days later, for instance on day 12, the cells can be split. The cells can be cultured and split again, for instance on day 16. Cells can be cultured for a suitable time period including but not limited to an additional 2-5,5-8, 8-12, 12-16, 16-20, 20-24, and 24-28 days for colony formation and clonal expansion.

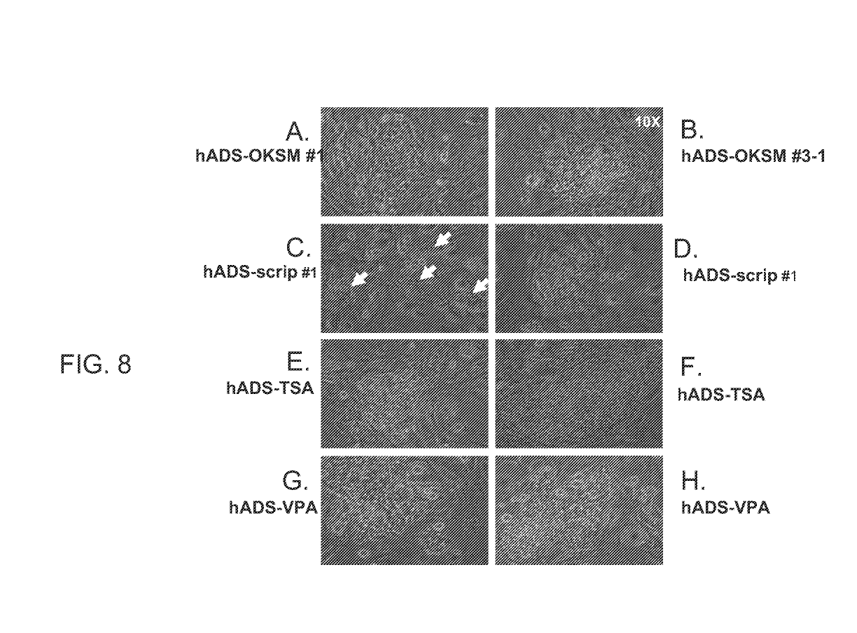

FIGS. 8A and 8B are photographs taken 9 days after viral infection of human adipose tissue derived adult stem cells transfected with lentivirus that over-expresses mouse Oct-4, Sox2, c-Myc, and Klf4. FIG. 8C and FIG. 8D are photographs taken 9 days after viral infection of human adipose tissue derived adult stem cells pre-treated with scriptaid. As shown in FIG. 8C, cells display morphologies consistent with embryonic stem cells or reprogrammed cells. ES cell-like colony formation was observed.

FIG. 8E and FIG. 8F are photographs taken 9 days after viral infection of human adipose tissue derived adult stem cells pre-treated with trichostatin A Again, multiple colonies displaying morphologies consistent with embryonic stem cells or reprogrammed cells were observed.

FIG. 8G and FIG. 8H are photographs taken 9 days after viral infection of human adipose tissue derived adult stem cells pre-treated with valproic acid. Colonies displaying characteristics of embryonic stem cells or reprogrammed cells were observed.

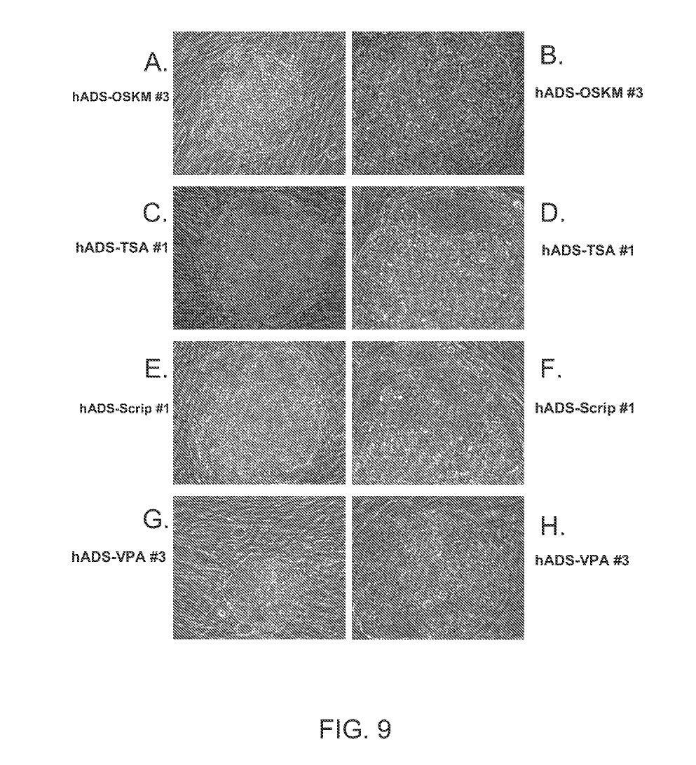

FIGS. 9A and 9B are photographs taken 11 days after viral infection of human adipose tissue derived adult stem cells transfected with lentivirus that over-expresses mouse Oct-4, Sox2, c-Myc, and Klf4. FIG. 9C and FIG. 9D represent photographs taken 11 days after viral infection of human adipose tissue derived adult stem cells pre-treated with trichostatin A. As shown in FIG. 9C, cells display morphologies consistent with embryonic stem cells or reprogrammed cells. ES cell-like colony formation was observed.

FIG. 9E and FIG. 9F are photographs taken 11 days after viral infection of human adipose tissue derived adult stem cells pre-treated with scriptaid. Again, multiple colonies displaying morphologies consistent with embryonic stem cells or reprogrammed cells were observed.

FIG. 9G and FIG. 9H are photographs taken 11 days after viral infection of human adipose tissue derived adult stem cells pre-treated with valproic acid. Colonies displaying characteristics of embryonic stem cells or reprogrammed cells were observed.

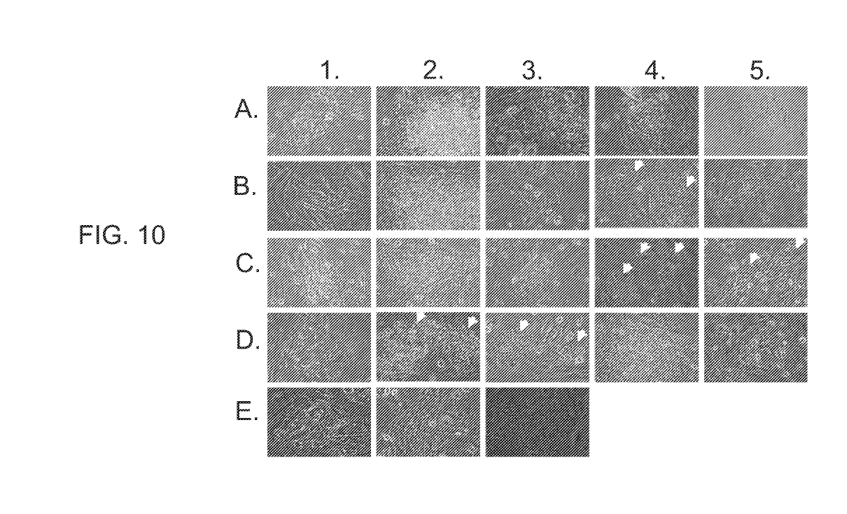

FIG. 10 is a panel of photographs of human adipose tissue derived adult stem cells pre-treated for five (5) days with trichostatin A (FIGI. 10, panels C1, C2, C3, C4, C5, D1, D2, D3, D4, D5, E1, E2, E3) or without pretreatment (FIG. 10, panels A1, A2, A3, A4, A5, B1, B2, B3, B4, B5) and then transfected with lentivirus that over-expresses mouse Oct-4, Sox2, c-Myc, and Klf4. The lentivirus was purchased from Millipore (Billerica, Mass.). Photographs show colony formation with morphologies consistent with embryonic stem cells. Cells that were pre-treated with TSA show about a two-fold increase in colony formation.

Theses results suggest that human adipose derived adult stem cells are am efficient and reliable source of cells for somatic cell reprogramming. The cells are reprogrammed efficiently and effectively. Reprogramming can be achieved in only 6˜7 days after delivery of four iPS factors with ˜2% reprogramming efficiency.

TSA treatment at a low concentration (1 μM) increased the somatic cell reprogramming efficiency by measuring the number of formed colonies. Numerical results are in Table VI. Epigenetic modification by pretreatment with an HDAC inhibitor is beneficial to increase the reprogramming efficiency.

TABLE VI
Number of ES cell-like colonies

| | No Treatment | TSA |
|---|---|---|
| First Experiment | 15 | 24 |
| Second Experiment | 12 | 21 |

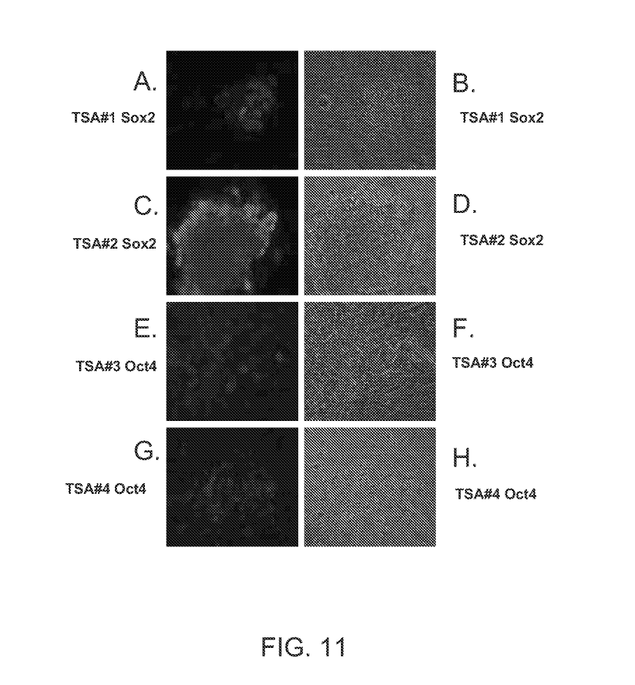

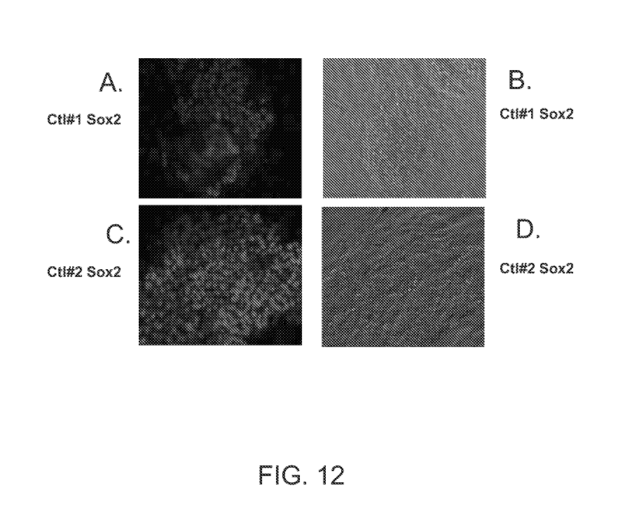

ES cell-like colonies, which resulted from pre-treatment of human adipose derived adult stem cells with TSA, showed Sox-2 and Oct4 expression (FIG. 11). FIG. 11A, 11C, 11E, and 11G show Sox-2 and Oct4 expression by immunohistochemistry. FIGS. 11B, 11D, 11F, and 11H show morphologies of the ES cell-like colonies. ES cell-like colonies from non pre-treatment show Sox-2 and Oct4 expression (FIG. 12A-12D).

These results demonstrate that colony formation and clonal expansion of colonies can be enhanced by pre-treatment of cell prior to transfection with the pluripotency genes. ES cell-like colony formation was about two-fold faster and much more efficient than traditional methods that have been reported. Furthermore, these data indicate that reprogramming efficiency is significantly enhanced by epigenetic modification through pre-treating cells with various HDAC inhibitors prior to a four-factor transfection.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated in their entirety by reference herein.

1. A method for reprogramming a cell comprising:
(a) delivering a transcription factor to a cell;
(b) exposing said cell to an agent that reduces the activity, expression, or activity and expression of a gene or protein involved in transcriptional repression; and
(c) selecting a cell, wherein differentiation potential has been restored to said cell.

2. The method of claim 1, wherein delivering a transcription factor to a cell comprises delivering a transcription factor selected from the group consisting of Oct-4, Sox-2, klf4, Nanog, Lin28 and c-myc.

3. The method of claim 1, wherein delivering a transcription factor to a cell comprises delivering Oct-4 and Sox-2.

4. The method of claim 1, wherein delivering a transcription factor to a cell comprises delivering no more than two transcription factors.

5. The method of claim 1, wherein delivering a transcription factor to a cell comprises delivering no more than three transcription factors.

6. The method of claim 1, wherein said agent is selected from the group consisting of an shRNA, an siRNA, an HDAC modulator, an HDAC inhibitor, a small molecule modulator, a methyl binding domain protein, a methyl adenosyltransferases (MAT), a DNA methyltransferases (DNMT), a histone methyltransferase, and a methyl cycle enzyme.

7. The method of claim 1 further comprising expanding said selected cell into a population of cells.

8. A method for reprogramming a cell comprising
(a) inducing expression of an endogenous transcription factor network within a cell having a first differentiation status;
(b) selecting a cell with a second differentiation status;
(c) expanding said selected cell into a population of cells.

9. The method of claim 8, wherein inducing the expression of an endogenous transcription factor comprises delivering a transcription factor to the cell.

10. The method of claim 8, wherein delivering a transcription factor to a cell comprises delivering a transcription factor selected from the group consisting of Oct-4, Sox-2, Klf4, Nanog, Lin28 and c-myc.

11. The method of claim 8, wherein inducing the expression of an endogenous transcription factor comprises exposing said cell to an agent that reduces the activity, expression, or activity and expression of a gene or protein involved in transcriptional repression.

12. The method of claim 11, wherein said agent is selected from the group consisting of an shRNA, an siRNA, an HDAC modulator, an HDAC inhibitor, a small molecule modulator, a methyl binding domain protein, a methyl adenosyltransferases (MAT), a DNA methyltransferases (DNMT), a histone methyltransferase, and a methyl cycle enzyme.

13. A method for reprogramming a cell comprising:
(a) culturing a cell in a medium comprising an agent that reduces the activity, expression, or activity and expression of a gene or protein involved in transcriptional repression;
(b) delivering a transcription factor to said cell after culturing in said medium of (a);
(c) selecting a cell with increased differentiation potential.

14. The method of claim 13, wherein said agent is selected from the group consisting of an shRNA, an siRNA, an HDAC modulator, an HDAC inhibitor, a small molecule modulator, a methyl binding domain protein, a methyl adenosyltransferases (MAT), a DNA methyltransferases (DNMT), a histone methyltransferase, and a methyl cycle enzyme

15. The method of claim 13, wherein delivering said transcription factor comprises delivering Oct-4 and Sox-2.

16. The method of claim 13, wherein delivering said transcription factor comprises delivering Oct-4, Sox2, Klf4, and c-myc.

17. The method of claim 13, wherein delivering said transcription factor comprises delivering Oct-4, Sox-2, Nanog and Lin28.

18. The method of claim 13 further comprising expanding said selected cell into a population of cells.

19. The method of claim 13, wherein said agent is an HDAC inhibitor.
